SEQUENCE LISTING (8 sequences)

SEQ ID NO: 1 - DNA, 19 bp, synthetic construct
  misc_feature 1..19: note = lonp1_forward
  source 1..19: mol_type = other DNA
tgccttgaaccctctctac

SEQ ID NO: 2 - DNA, 20 bp, synthetic construct
  misc_feature 1..20: note = lonp1_reverse
  source 1..20: mol_type = other DNA
tctgcttgatcttctcctcc

SEQ ID NO: 3 - DNA, 19 bp, synthetic construct
  misc_feature 1..19: note = mthsp70_forward
  source 1..19: mol_type = other DNA
actcctccatttatccgcc

SEQ ID NO: 4 - DNA, 21 bp, synthetic construct
  misc_feature 1..21: note = mthsp70_reverse
  source 1..21: mol_type = other DNA
acctttgcttgtttaccttcc

SEQ ID NO: 5 - DNA, 19 bp, synthetic construct
  misc_feature 1..19: note = hsp60_forward
  source 1..19: mol_type = other DNA
acctgctcttgaaattgcc

SEQ ID NO: 6 - DNA, 21 bp, synthetic construct
  misc_feature 1..21: note = hsp60_reverse
  source 1..21: mol_type = other DNA
caatccctcttctccaaacac

SEQ ID NO: 7 - DNA, 20 bp, synthetic construct
  misc_feature 1..20: note = actb_forward
  source 1..20: mol_type = other DNA
tgtttgagaccttcaacacc

SEQ ID NO: 8 - DNA, 18 bp, synthetic construct
  misc_feature 1..18: note = actb_reverse
  source 1..18: mol_type = other DNA
atgtcacgcacgatttcc

(19) United States
(12) Patent Application Publication
(10) Pub. No.: US 2026/0029412 A1
(43) Pub. Date: Jan. 29, 2026

(54) COMPOSITIONS AND METHODS FOR TREATING CANCER BY INHIBITING MITOCHONDRIAL STRESS-INDUCED PROTEIN CARBOXYL-TERMINAL ALANINE AND THREONINE TAILING (MSICAT-TAILING)

(71) Applicants: Southern Methodist University, Dallas, TX (US); The Regents of the University of California, Oakland, CA (US)

(72) Inventors: Zhihao Wu, Dallas, TX (US); Bei Zhang, Dallas, TX (US); Ting Cai, Dallas, TX (US); Rongze Lu, San Francisco, CA (US)

(21) Appl. No.: 19/283,519

(22) Filed: Jul. 29, 2025

Related U.S. Application Data
(60) Provisional application No. 63/676,513, filed on Jul. 29, 2024.

(51) Int. Cl. G01N 33/68; A61K 31/365; A61K 31/40; A61K 31/7076; A61K 31/7088; G01N 33/50

(52) U.S. Cl. CPC G01N 33/6893; A61K 31/365; A61K 31/40; A61K 31/7076; A61K 31/7088; G01N 33/5005; G01N 2333/4703; G01N 2800/7028

(57) ABSTRACT

Provided herein are compositions and methods for detecting presence of cancer cells, and treating the cancer cells, in a human, the method comprising: obtaining, or having obtained, a human tissue sample with cancer cells; and detecting in the human tissue sample for abnormalities in ribosome-associated quality control by detecting the presence of mitochondrial stress-induced protein carboxyl-terminal terminal alanine and threonine tailing (msiCAT-tailing) proteins, wherein the presence of msiCAT-tailing is indicative of the presence of cancer stem cells.

### CROSS-REFERENCE TO RELATED APPLICATIONS

This application claims priority to U.S. Provisional Application Ser. No. 63/676,513, filed Jul. 29, 2024, the entire contents of which is incorporated herein by reference.

### STATEMENT OF FEDERALLY FUNDED RESEARCH

This invention was made with government support under R35GM150190 awarded by the National Institutes of Health. The government has certain rights in the invention.

### TECHNICAL FIELD OF THE INVENTION

The present invention relates in general to the field of cancer treatments, and more particularly, to novel compositions and methods for treating cancer by inhibiting mitochondrial stress-induced protein carboxyl-terminal alanine and threonine tailing (msiCAT-tailing).

### INCORPORATION-BY-REFERENCE OF MATERIALS FILED ON COMPACT DISC

The present application includes a Sequence Listing which has been submitted in XML format via EFS-Web and is hereby incorporated by reference in its entirety. Said XML copy, created on Jul. 28, 2025, is named SMU1064.xml and is 11,103 bytes in size.

### BACKGROUND OF THE INVENTION

Without limiting the scope of the invention, its background is described in connection with glioblastoma.

One such invention is described in U.S. Pat. No. 12,000,834, issued to Cumba-Garcia and Parney, entitled, “Methods and Materials for Assessing and Treating Cancer”. These inventors as said to teach methods and materials for identifying and/or treating mammals having cancer in which small extracellular vesicles (EVs), such as exosomes, are obtained from a mammal suspected of having a cancer can be used to identify the mammal as having cancer and to treat the mammal. Treatments can include surgery, radiation therapy, chemotherapy, tumor treating fields (TTF) therapy, targeted therapy, hormone therapy, angiogenesis inhibitor therapy, tumor vaccination, checkpoint blockade therapy, and any combinations thereof.

Another such invention is described in U.S. Pat. No. 11,998,543, issued to Kang and Kim, entitled, “Pharmaceutical Composition Comprising Aldehyde Inhibitor and Anticancer Agent for Treatment of Brain Cancer”. The inventors are said to teach a biguanide-based compound, such as phenformin, to treat and improve the prognosis of brain cancer, kill cancer cells and inhibiting cancer stem cell.

Another such invention is described in U.S. Pat. No. 11,998,540, issued to Liau, et al., entitled “Compositions and Methods For Treating Drug-tolerant Glioblastoma”. These inventors are said to teach compositions and methods for the treatment of drug-tolerant glioblastoma, and in particular glioblastoma stem cells, by contacting a glioblastoma stem cell with a platelet-derived growth factor receptor alpha inhibitor and one or more of a histone lysine demethylase inhibitor and a Notch inhibitor.

Despite these advances, a need remains for novel compositions and methods for detecting cancer stem cells, reducing or eliminating the cancer cells, and making cancer cells that have become resistant to cancer drugs susceptible to the same.

### SUMMARY OF THE INVENTION

As embodied and broadly described herein, an aspect of the present disclosure relates to a method for detecting presence of cancer cells in a human tissue sample, the method comprising: obtaining, or having obtained, a human tissue sample with cancer cells; and detecting in the human tissue sample for abnormalities in ribosome-associated quality control by detecting the presence of mitochondrial stress-induced protein carboxyl-terminal terminal alanine and threonine tailing (msiCAT-tailing) proteins, wherein the presence of msiCAT-tailing is indicative of the presence of cancer stem cells. In one aspect, the cancer stem cells are glioma, glioblastoma, medulloblastoma, oligodendroglioma, or ependymoma stem cells, or combinations thereof. In another aspect, the method further comprises detecting an increase in expression of at least one of: ABCE1, ASCC1, ASCC2, ASCC3, RACK1, or VCP. In another aspect, the method further comprises detecting a decrease in expression of ANKZF1. In another aspect, the msiCAT-tailing proteins are msiCAT-tailed mitochondrial proteins are selected from C-I30, COX4 (cytochrome c oxidase subunit 4), and ATP5α (ATP synthase F1 subunit alpha). In another aspect, the method further comprises detecting an increase in expression or post-translational modification of nuclear export mediator factor (NEMF), or both. In another aspect, the msiCAT-tailing proteins are msiCAT-tailed mitochondrial proteins detected by at least one of: measuring a presence of detergent insoluble aggregates; measuring a presence of oligomers; measuring msiCAT-tailing modification of mitochondrial ATP synthase F1 subunit alpha (ATP5α); measuring an increase in cytoplasmic ATP5α-AT20 near mitochondria and forming protein aggregates; measuring a higher mitochondrial membrane potential; measuring a reduced ATP production; measuring a reduction in open mitochondrial permeability transition pores (MPTP); measuring overexpression of short AT repeat tail (AT3); measuring overexpression of long AT repeat tail (AT20) fused with ATP5α; or determining an increase in a ratio of cells that have aggregates versus cells that do not have aggregates. In another aspect, the method further comprises measuring a rate of protein translation in the cancer and detecting a decrease in the rate of protein translation, wherein a decrease in the rate of protein translation is indicative of the presence of glioblastoma stem cells in the human tissue sample. In another aspect, the method further comprises measuring ATP5α-AT20 proteins, wherein an increase in expression of ATP5α-AT20 proteins is indicative of the presence of glioblastoma stem cells in the human tissue sample. In another aspect, the method further comprises determining if glioblastoma cancer is resistant to staurosporine (STS), temozolomide (TMZ), or both.

As embodied and broadly described herein, an aspect of the present disclosure relates to a method for treating a mammal having a cancer with abnormalities in ribosome-associated quality control, wherein the method comprises: identifying in the cancer abnormalities in ribosome-associated quality control by detecting a presence of mitochondrial stress-induced protein carboxyl-terminal terminal alanine and threonine tailing (msiCAT-tailing); and administering a cancer treatment to the mammal having the cancer, wherein the cancer treatment is selected from the group consisting of a nucleic acid msiCAT-tailing inhibitor or an msiCAT-tailing antagonist, in an amount effective to reduce the presence of msiCAT-tailing. In one aspect, the cancer is glioma, glioblastoma, medulloblastoma, oligodendroglioma, ependymoma, or combinations thereof. In another aspect, the msiCAT-tailing antagonist is selected from at least one of: anisomycin, puromycin, borrelidin, isomers and derivatives thereof, and combinations of the same. In another aspect, the msiCAT-tailing antagonist is selected from anisomycin, isomers and derivatives thereof. In another aspect, the nucleic acid CAT-tailing inhibitor at least one of: knocks down NEMF (sgNEMF) expression, or increase the expression of ANKZF1 (oeANZKF1). In another aspect, the cancer is resistant to staurosporine (STS), temozolomide (TMZ), or both. In another aspect, the treatment reduces migration of glioma, glioblastoma, medulloblastoma, oligodendroglioma, or ependymoma stem cells, or combinations thereof.

As embodied and broadly described herein, an aspect of the present disclosure relates to a method for treating a cancer that has become resistant to staurosporine (STS), temozolomide (TMZ), or both, wherein the method comprises: administering a mitochondrial stress-induced protein carboxyl-terminal terminal alanine and threonine tailing (msiCAT-tailing) antagonist in an amount sufficient to render the cancer susceptible to the staurosporine (STS), temozolomide (TMZ), or both. In one aspect, the cancer is glioma, glioblastoma, medulloblastoma, oligodendroglioma, ependymoma, or combinations thereof. In another aspect, the msiCAT-tailing antagonist is selected from at least one of: anisomycin, puromycin, borrelidin, isomers and derivatives thereof, and combinations of the same. In another aspect, the msiCAT-tailing antagonist is selected from anisomycin, isomers and derivatives thereof. In another aspect, the msiCAT-tailing antagonist is a nucleic acid CAT-tailing inhibitor that at least one of: knocks down NEMF (sgNEMF) expression, or increase the expression of ANKZF1 (oeANZKF1).

As embodied and broadly described herein, an aspect of the present disclosure relates to a method for treating a cancer with abnormalities in ribosome-associated quality control, wherein the method comprises administering a cancer treatment to a mammal having cancer and identified as having cancer are resistant to staurosporine (STS), temozolomide (TMZ), or both, comprising: obtaining, or having obtained, a human tissue sample with cancer having the abnormalities in ribosome-associated quality control; detecting in the human tissue sample for abnormalities in ribosome-associated quality control by detecting a presence of mitochondrial stress-induced protein carboxyl-terminal terminal alanine and threonine tailing (msiCAT-tailing) proteins, wherein the presence of msiCAT-tailing is indicative of the presence of glioblastoma stem cells; and administering a cancer treatment to the mammal having cancer, wherein the cancer treatment is selected from the group consisting of a nucleic acid msiCAT-tailing inhibitor or a msiCAT-tailing antagonist, in an amount effective to reduce the presence of msiCAT-tailing. In one aspect, the cancer is glioma, glioblastoma, medulloblastoma, oligodendroglioma, or ependymoma, or combinations thereof. In another aspect, the msiCAT-tailing antagonist is selected from at least one of: anisomycin, puromycin, borrelidin, isomers and derivatives thereof, and combinations of the same. In another aspect, the msiCAT-tailing antagonist is selected from anisomycin, isomers and derivatives thereof. In another aspect, the nucleic acid msiCAT-tailing inhibitor at least one of: knocks down NEMF (sgNEMF) expression, or increase the expression of ANKZF1 (oeANZKF1). In another aspect, the cancer is resistant to staurosporine (STS), temozolomide (TMZ), or both, and the msiCAT-tailing inhibitor increases susceptibility of the cancer to STS or MTZ treatment. In another aspect, the treatment reduces migration of glioma, glioblastoma, medulloblastoma, oligodendroglioma, or ependymoma stem cells, or combinations thereof.

### BRIEF DESCRIPTION OF THE DRAWINGS

For a more complete understanding of the features and advantages of the present invention, reference is now made to the detailed description of the invention along with the accompanying figures and in which:

FIGS. 1A to 1F are evidence for msiCAT-tailing on mitochondrial proteins in GBM cells. (FIG. 1A) RQC gene expression levels in GBM tumor tissues (n=153) compared to normal brain tissues (n=206) (unpaired Student's t-test; *, log FC (fold change)>1; adj.P.Val<0.001). (FIG. 1B) Western blot analysis of msiCAT-tailed mitochondrial proteins and RQC factors in patient-derived GSC and control NSC cells, using ACTIN as the loading control. Arrowheads indicate short CAT-tailed mitochondrial proteins; “short” and “long” refer to exposure time; the numbers represent fold changes compared to controls (NSC). (FIG. 1C) Western blot of 5×FLAG-tagged β-globin reporter proteins in GBM and control cells, showing more CAT-tailed proteins in GBM cells, using ACTIN as the loading control. The numbers represent fold changes compared to controls (NHA without any treatment); the purple numbers represent the ratio of top bracket (CAT-tailed) to bottom bracket (non-CAT-tailed) sections. (FIG. 1D) Western blot of overexpressed ATP5α-AT3 and ATP5α-AT20 in GBM and control cells, using GAPDH as the loading control; arrowheads indicate endogenous ATP5α, ATP5α-AT3, ATP5α-AT20, and oligomers/aggregates of msiCAT-tailed ATP5α proteins. The purple numbers represent the ratio of dark (exogenous) to light (endogenous) sections. (FIG. 1E) Immunofluorescence staining shows endogenous ATP5α protein aggregates in GBM cells, with TOM20 (dark) as a mitochondrial marker. Arrows indicate ATP5α protein aggregates. (FIG. 1F) Quantification of E (n=3; chi-squared test; ***, P<0.001; ****, P<0.0001); the total number of cells counted is indicated in the columns.

FIGS. 2A to 2L show the impact of msiCAT-tailed ATP5α proteins on mitochondrial functions in GBM cells. (FIG. 2A) TMRM staining shows a high mitochondrial membrane potential in patient-derived GSC cells (n=3; unpaired Student's t-test; ***, P<0.001; ****, P<0.0001). (FIG. 2B) ATP measurement shows a low mitochondrial ATP production in patient-derived GSC cells (n=3; unpaired Student's t-test; **, P<0.01; ***, P<0.001). (FIG. 2C, FIG. 2D) JC-10 staining reveals a reduced mitochondrial membrane potential in GBM cells, but not in NHA control cells, upon both genetic (FIG. 2C) and pharmacological (FIG. 2D) inhibition of the msiCAT-tailing pathway (n=3; unpaired Student's t-test; ***, P<0.001; ****, P<0.0001; ns, not significant). (FIG. 2E) JC-10 staining reveals an increased mitochondrial membrane potential in GBM cells, but not in control cells, upon overexpression of ATP5α-AT3 and ATP5α-AT20 (n=3; unpaired Student's t-test; ****, P<0.0001; ns, not significant). (FIG. 2F) Western blot of FLAG-tagged ATP5α, NEMF, and ANKZF1 in GBM cells and control cells, using ACTIN as the loading control. (FIG. 2G) JC-10 staining reveals an increased mitochondrial membrane potential in GBM cells, but not in NHA control cells, upon overexpression of ATP5α-AT3 and ATP5α-AT20 with concurrent genetic inhibition of the endogenous msiCAT-tailing pathway (n=3; unpaired Student's t-test; *, P<0.05; **, P<0.01). (FIG. 2H) BN-PAGE western blot of ATP5α and Flag shows that ATP5α-AT3 is incorporated into the mitochondrial Complex-V (ATP synthase), while ATP5α-AT20 forms high molecular weight protein aggregates in GBM cells. SC: respiratory supercomplex; C-V: Complex-V/ATP synthase. (FIG. 2I, FIG. 2K) Oxygen consumption rate (OCR) data indicate a reduction in mitochondrial oxygen consumption in SF268 cells expressing ATP5α-AT3 and ATP5α-AT20. Oligomycin (1.5 μM), FCCP (1.0 μM), and rotenone/antimycin A (R/A, 0.5 μM) were sequentially added. (FIG. 2J, FIG. 2L) Statistics of mitochondrial respiration parameters in (FIG. 2I, FIG. 2K), including non-mitochondrial respiration, basal respiration, maximum respiration, spare respiration, proton leaks, and ATP production (n=3; unpaired Student's t-test; *, P<0.05; **, P<0.01; ***, P<0.001; ****, P<0.0001; ns, not significant).

FIGS. 3A to 3M shows that the msiCAT-tailing product regulates MPTP status in GBM cells.

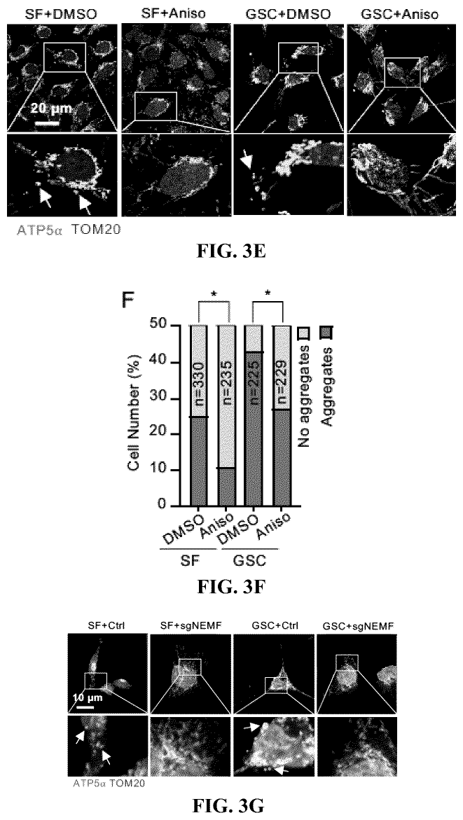

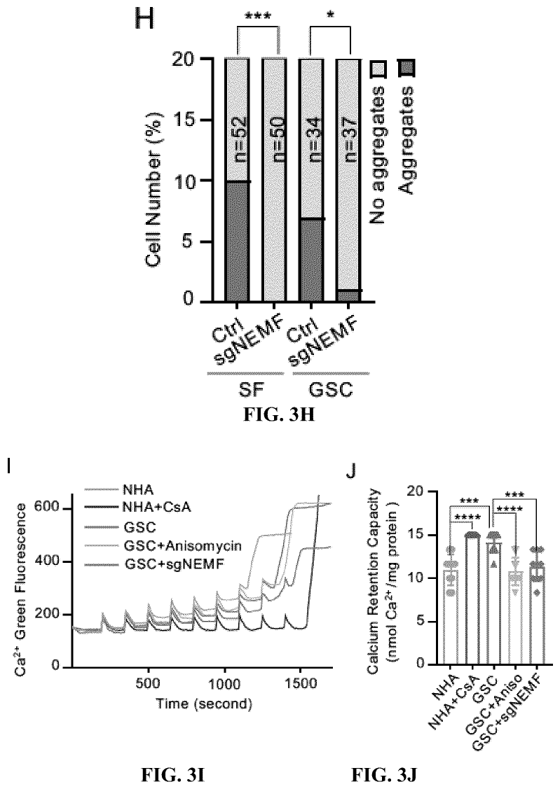

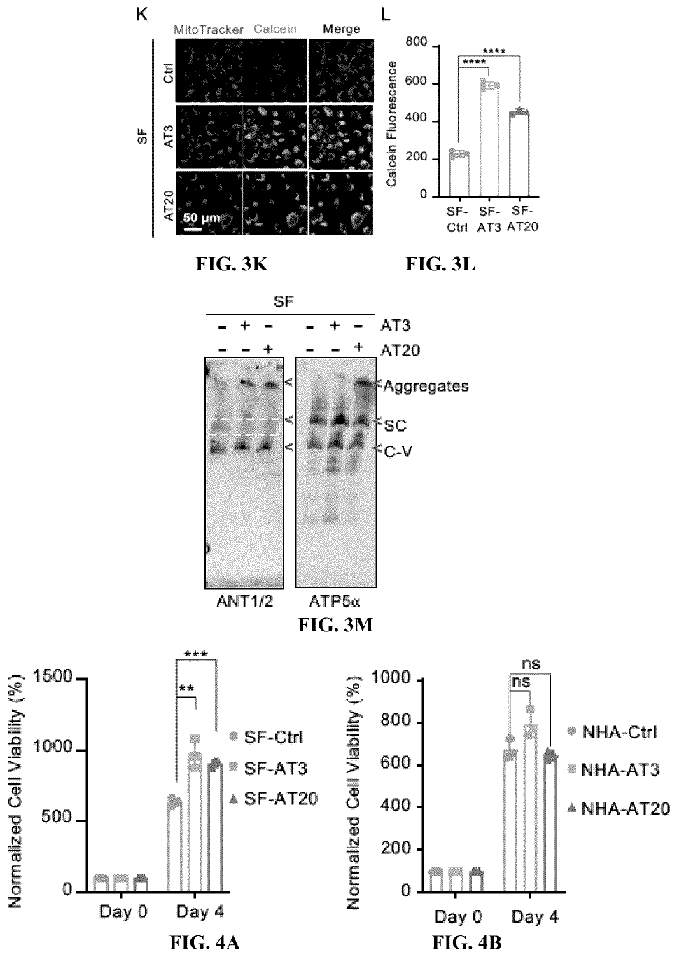

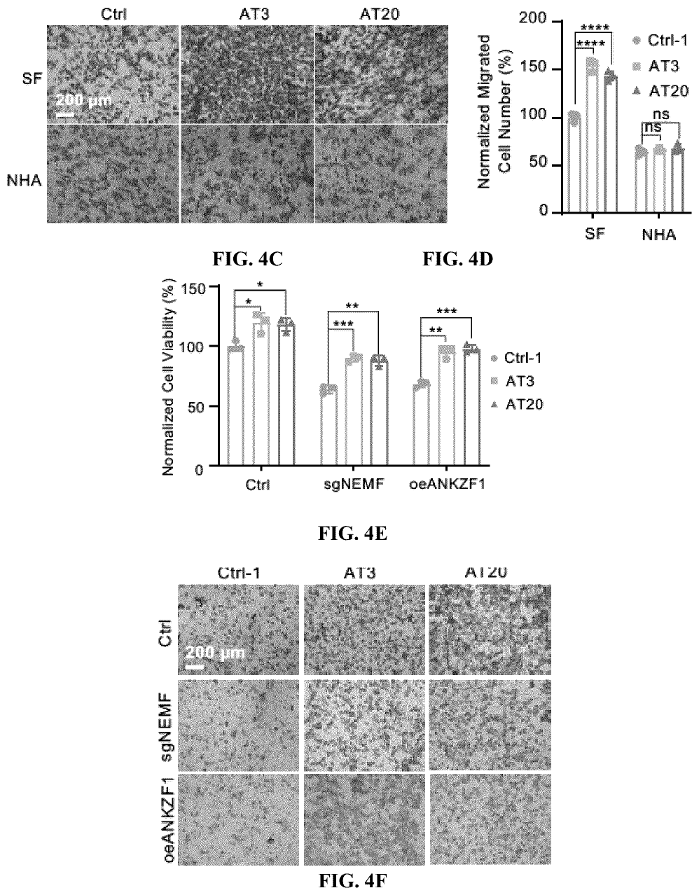

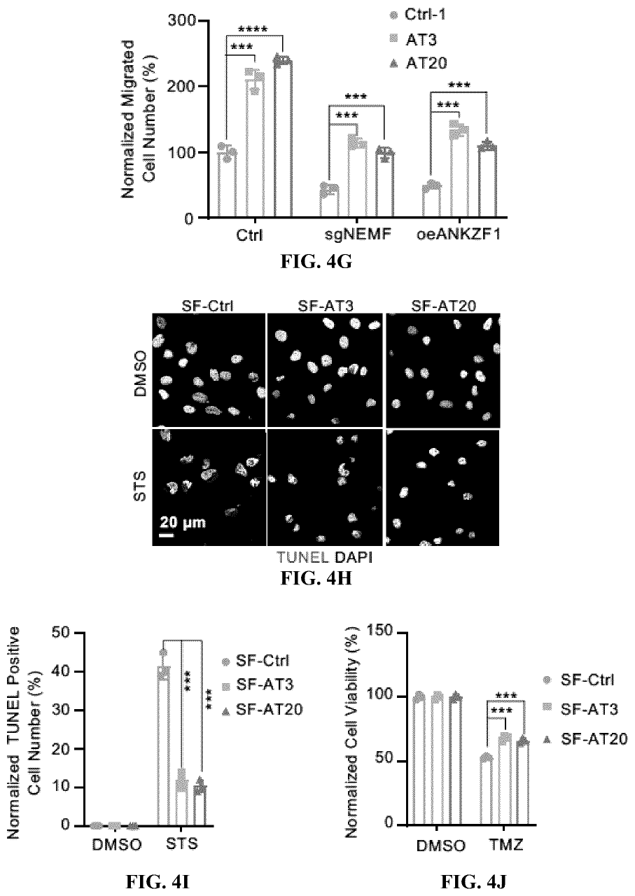

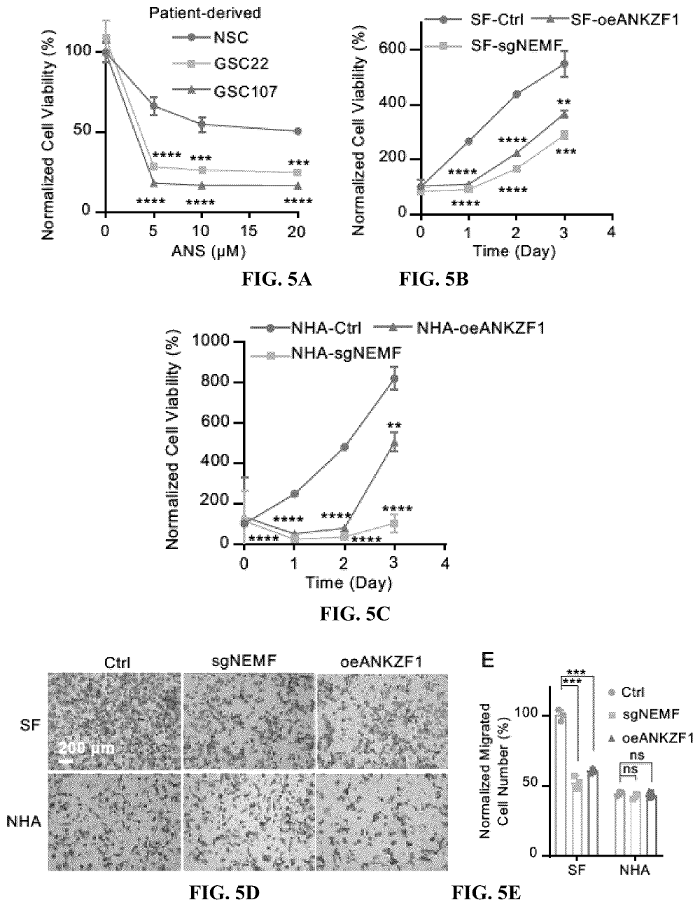

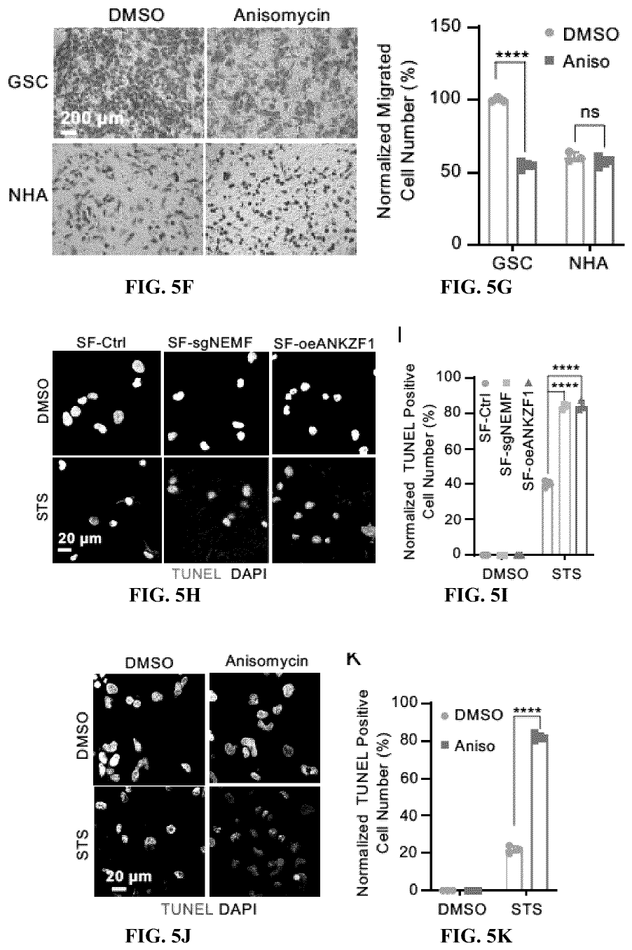

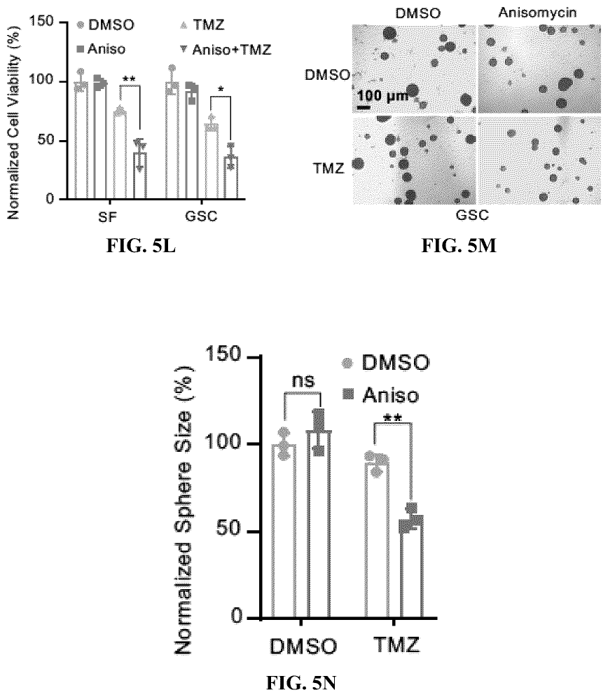

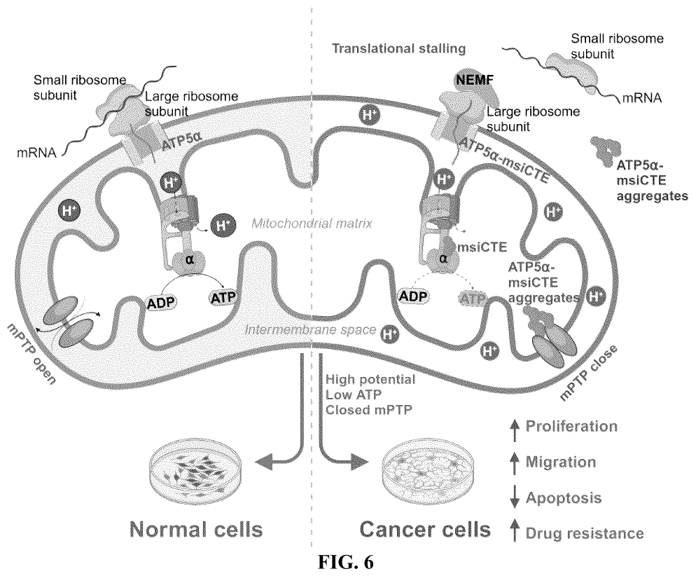

(FIG. 3A, FIG. 3C) MPTP activity assay shows reduced mitochondrial permeability transition pore opening in GSC cells compared to NHA (control) cells. Pharmacological (FIG. 3A, anisomycin 200 nM) and genetic (sgNEMF) inhibition of CAT-tailing reverse it. (FIG. 3B, FIG. 3D) Quantification of (FIG. 3A, FIG. 3C) (n=3; unpaired Student's t-test; ****, P<0.0001; ns, not significant). (FIG. 3E, FIG. 3G) Immunofluorescence staining shows that anisomycin treatment (FIG. 3E) and sgNEMF (FIG. 3G) inhibit endogenous ATP5α protein aggregation in GBM cells, using TOM20 (red) as a mitochondrial marker. (FIG. 3F, FIG. 3H) Quantification of (FIG. 3E, FIG. 3G) (n=3; chi-squared test; *, P<0.05; ***, P<0.001); the total number of cells counted is indicated in the columns. (FIG. 3I) Calcium retention capacity (CRC) assay of isolated mitochondria, measured with Calcium Green-5N dye, shows high CRC in GBM cells compared to control NHA cells. CsA (Cyclosporin A, MPTP inhibitor) serves as a positive control. (FIG. 3J) Statistic of (FIG. 3I) shows attenuated CRC in mitochondria pretreated with anisomycin or with sgNEMF (n=3; unpaired Student's t-test; ***, P<0.001; ****, P<0.0001). (FIG. 3K) MPTP activity assay shows that ectopic expression of ATP5α-AT3 and ATP5α-AT20 inhibits MPTP opening in GBM cells. (FIG. 3L) Quantification of (K) (n=3; unpaired Student's t-test; ****, P<0.0001). (FIG. 3M) BN-PAGE western blot shows that ATP5α-AT3 and ATP5α-AT20 expression alters ANT1/2 protein patterns in GBM cells, resulting in a missing band (circled in yellow dashed line), and formation of high molecular weight aggregates. SC: respiratory supercomplex; C-V: Complex V/ATP synthase.

FIGS. 4A to 4J show that the msiCAT-tailed ATP5α protein promotes GBM progression. (FIG. 4A) MTT assay indicates increased proliferation caused by ATP5α-AT3 and ATP5α-AT20 expression in GBM cells (n=3; unpaired Student's t-test; **, P<0.01; ***, P<0.001). (FIG. 4B) MTT assay indicates no change in proliferation caused by ATP5α-AT3 and ATP5α-AT20 expression in NHA cells (n=3; unpaired Student's t-test; ns, not significant). (FIG. 4C) Transwell assay reveals enhanced migration induced by ATP5α-AT3 and ATP5α-AT20 expression in GBM (SF) cells but not in control (NHA) cells. (FIG. 4D) Quantification of (FIG. 4C) shows the number of migrated cells (n=3; unpaired Student's t-test; ****, P<0.0001; ns, not significant). (FIG. 4E) MTT assay indicates an increased proliferation in GBM cells, upon overexpression of ATP5α-AT3 and ATP5-AT20 with concurrent genetic inhibition of the endogenous msiCAT-tailing pathway (n=3; unpaired Student's t-test; *, P<0.05; **, P<0.01). (FIG. 4F) Transwell assay reveals enhanced migration upon overexpression of ATP5α-AT3 and ATP5α-AT20 with concurrent genetic inhibition of the endogenous msiCAT-tailing pathway. (FIG. 4G) Quantification of (FIG. 4F) shows the number of migrated cells (n=3; unpaired Student's t-test; ***, P<0.001; ****, P<0.0001). (FIG. 4H) TUNEL staining shows that staurosporine (STS, 1 μM)-induced apoptosis is attenuated by ATP5α-AT3 and ATP5α-AT20 expression in GBM cells, using TUNEL-Cy3 as an apoptotic cell indicator and DAPI as a nucleus indicator. (FIG. 4I) Quantification of (FIG. 4H) shows the percentage of TUNEL-positive cells in the population (n=3; unpaired student's t-test; ***, P<0.001), using DMSO as the vehicle control. (FIG. 4J) MTT assay indicates an enhanced resistance to temozolomide (TMZ, 150 PM) induced by ATP5α-AT3 and ATP5α-AT20 expression. The TMZ-treated/SF-Ctrl group is used as the control (n=3; unpaired Student's t-test; ***, P<0.001).

FIGS. 5A to 5N shows that inhibition of msiCAT-tailing impedes GBM progression. (FIG. 5A) Cell viability assay shows greater sensitivity to anisomycin treatment in patient-derived GSC cells than control NSC cells at 48 hours (n=3; unpaired Student's t-test; ***, P<0.001; ****, P<0.0001; compared to controls at the corresponding dose). (FIG. 5B) MTT assay indicates reduced GBM cell proliferation by genetic inhibition of the msiCAT-tailing pathway (n=3; unpaired Student's t-test; **, P<0.01; ***, P<0.001; ****, P<0.0001, compared to controls at the corresponding time). (FIG. 5C) MTT assay indicates reduced NHA cell proliferation by genetic inhibition of the msiCAT-tailing pathway (n=3; unpaired Student's t-test; **, P<0.01; ****, P<0.0001, compared to controls at the corresponding time). (FIG. 5D, FIG. 5F) Transwell assay reveals that both genetic (FIG. 5D) and pharmacological (FIG. 5F) inhibition of the msiCAT-tailing pathway hampers the migration of GBM cells but not control cells. (FIG. 5E, FIG. 5G) Quantification of (FIG. 5D, FIG. 5F) showing the number of migrated cells (n=3; unpaired Student's t-test; ***, P<0.001; ****, P<0.0001; ns, not significant). (FIG. 5H, FIG. 5J) TUNEL staining shows that both genetic (FIG. 5H) and pharmacological (FIG. 5J) inhibition of the msiCAT-tailing pathway enhances STS-induced apoptosis in GBM cells, using TUNEL-Cy3 as an apoptotic cell indicator and DAPI as a nucleus indicator. (FIG. 5I, FIG. 5K) Quantification of (FIG. 5H, FIG. 5J) showing the percentage of TUNEL-positive cells in the population (n=3; unpaired Student's t-test; ****P<0.0001), using DMSO as the vehicle control. (FIG. 5L) MTT assay shows that pharmacological inhibition of the msiCAT-tailing pathway decrease the resistance of GBM cells to temozolomide (TMZ, 150 PM) treatment (n=3; unpaired Student's t-test; *P<0.05; **, P<0.01). (FIG. 5M) Neurosphere formation assay shows that reduced spheroid formation caused by pharmacological inhibition of the msiCAT-tailing pathway can synergize with TMZ in GBM cells. (FIG. 5N) Quantification of (M) (n=3; unpaired Student's t-test; **, P<0.01).

FIG. 6 is a diagram that shows the impact of msiCAT-tail modified ATP5α protein on mitochondrial function in GBM cells. In healthy cells, ATP5α protein, encoded by the nuclear genome, is imported into the mitochondrial matrix via the TOM/TIM complex through co-translational import and incorporated into ATP synthase (Left). Conversely, in GBM cells, the CAT-tailed ATP5α protein can either form aggregates near the mitochondrial outer membrane or be imported into the mitochondria. Within the mitochondrial matrix, proteins with shorter CAT-tails readily integrate into ATP synthase, disrupting its functionality. This dysfunction is characterized by a reduced ATP synthesis rate and proton (H⁺) accumulation, leading to an elevated mitochondrial membrane potential (ΔΨm). These alterations in ATP synthase ultimately trigger malfunction of the MPTP, consequently affecting cell proliferation, migration, and resistance to drug-induced apoptosis (Right).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the phrase “abnormalities in ribosome-associated quality control” refer to cells in which accelerated protein synthesis and disordered cell metabolism leads to ribosome collisions caused by frequent translation stalls. Such abnormalities in ribosome-associated quality control have been found in cancer or cancer cells, due to the rapid and sustained proliferation of the cancer or cancer cells, which require accelerated protein synthesis.

As used herein, the phrase “mitochondrial stress-induced protein carboxyl-terminal terminal alanine and threonine tailing (msiCAT-tailing) proteins” refers to the modification of the nascent peptide chains through the C-terminal alanine and threonine (CAT-tailing) mechanism to degrade aberrant peptides by the Ltn1/VCP/NEMF complex. CAT-tails can push lysine residues out of the ribosome exit channel to achieve Ltn1-mediated ubiquitination, promote degradation of faulty nascent peptide chains. However, CAT-tailed proteins are also prone to form detergent-insoluble aggregates.

As used herein, a “nucleic acid CAT-tailing inhibitor” refers to an RNA, DNA, or mimics thereof, that interfere with the expression or formation of short tail (ATP5α-AT3) and long tail (ATP5α-AT20). Nucleic acids can also modify expression of NEMF (e.g., using an sgNEMF), or overexpressing ANKZF1 (oeANZKF1). The nucleic acid CAT-tailing inhibitor may be selected from at least one of: a DNA plasmid, a small interfering RNA (siRNA), a messenger RNA (mRNA), a guide RNA (gRNA), a prime editing guide RNA (peg RNA), a CRISPR RNA (crRNA), a trans-activating CRISPR RNA (tracrRNA), a circular RNA, a microRNA (miRNA), a primary miRNA (pri-miRNA), a precursor miRNA (pre-miRNA), a piwi-interacting RNA (piRNA), a transfer RNA (tRNA), a long noncoding RNA (lncRNA), an antisense oligonucleotide (ASO), a short hairpin RNA (shRNA), a small activating RNA (saRNA), a small nucleolar RNAS (snoRNA), a gapmer, a locked nucleic acid (LNA), a peptide nucleic acid (PNA), an expression vector, a circular DNA, a linear double stranded DNA, an RNA plasmid, a DNA minicircle, a dumbbell-shaped DNA minimal vector and an RNA minicircle. The nucleic acid CAT-tailing inhibitor can be provided exogenously, such as in the form of aptamers or other nucleic acids which may or may not be modified to reduce degradation by nucleases. The nucleic acid CAT-tailing inhibitor can be expressed endogenously using liposomal nucleic acids, or transcribed from a vector that endogenously expresses the nucleic acid CAT-tailing inhibitor in the cancer cell. Non-limiting examples of such vectors include, e.g., a gene therapy vector selected from the group consisting of an adeno-associated virus (AAV) vector, an adenovirus vector, a lentivirus vector, a retrovirus vector, a herpes virus, a SV40 vector, a poxvirus vector, and combinations thereof.

As used herein, a “CAT-tailing antagonist” refers to molecules that reduce or eliminate CAT-tailing. Non-limiting examples of CAT-tailing antagonists include, e.g., anisomycin, puromycin, borrelidin, isomers and derivatives thereof, and combinations of the same.

As used herein, a “pharmaceutically acceptable” component is one that is suitable for use with humans and/or animals without undue adverse side effects (such as toxicity, irritation, and allergic response) commensurate with a reasonable benefit/risk ratio.

While it is possible to administer the active ingredient alone, it is common to present it as part of a pharmaceutical formulation. These formulations comprise the pharmacological agent in a therapeutically or pharmaceutically effective dose together with one or more pharmaceutically or therapeutically acceptable carriers and optionally other therapeutic ingredients. Various considerations are described, e.g., in Gilman et al. (eds) (1990) Goodman And Gilman's: The Pharmacological Bases Of Therapeutics, 8th Ed., Pergamon Press; Novel Drug Delivery Systems, 2nd Ed., Norris (ed.) Marcel Dekker Inc. (1989), and Remington's Pharmaceutical Sciences, relevant portions incorporated herein by reference. Methods for administration are discussed therein, e.g., for oral, intravenous, intraperitoneal, or intramuscular administration, and others. Pharmaceutically acceptable carriers will include water, saline, buffers, and other compounds described, e.g., in the Merck Index, Merck & Co., Rahway, N.J., or Bioreversible Carriers In Drug Design, Theory And Application, Roche (ed.), Pergamon Press, (1987).

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, to shrink or not metastasize. The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, “cancer” refers to all types of cancer or neoplasm or malignant tumors found in mammals, including glioblastoma. Examples of cancers are cancer of the brain, breast, cervix, colon, head and neck, kidney, lung, non-small cell lung, melanoma, mesothelioma, ovary, sarcoma, stomach, uterus and medulloblastoma. For example, brain cancers selected from glioma, glioblastoma, medulloblastoma, oligodendroglioma, and ependymoma.

Anisomycin (1), isomers and derivatives thereof can be selected from at least one of:

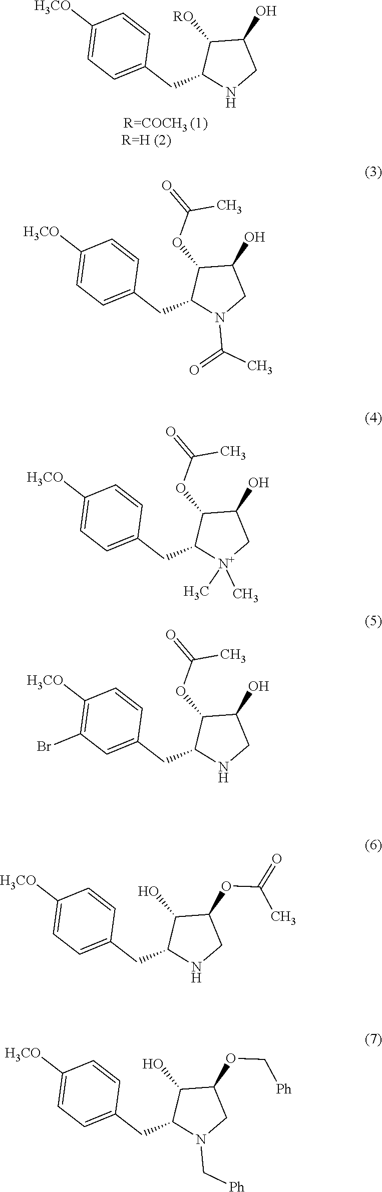

See: Ngyuen, et al., Systematic Exploration of Functional Group Relevance for Anti-Leishmanial Activity of Anisomycin, Biomedicines 2023, 11(9), 2541; doi.org/10.3390/biomedicines11092541.

Excessive protein synthesis is particularly important in rapidly dividing cells, such as cancer cells. To cope with this demand, cancer cells extensively regulate the initiation, elongation and termination of their protein translation (1). However, increased protein translation also proportionally accumulates the likelihood of translation errors (2). Combined with the significant changes in cellular metabolism, such as energy fluctuations and redox state shifts, how to respond to various sudden abnormal events during the protein translation process becomes essential. Ribosome-associated quality control (RQC) is a recently discovered class of mechanisms in eukaryotic cells that sense and rescue ribosome decelerations, stalls, and even collisions that occur during translation elongation or termination steps (3, 4).

RQC consists of a sequence of molecular events. The first is that the ZNF598/RACK1 complex recognizes the characteristic 40S-40S interface formed by collided ribosomes and promotes the ubiquitination of specific 40S subunit proteins (5, 6). Subsequently, the ASC-1 complex dissociates the leading collided ribosome (7, 8). Events that occur thereafter include: ribosomal subunit splitting and recycling (9), modification of the nascent peptide chains through the C-terminal alanine and threonine (CAT-tailing) mechanism (10), release of CAT-tailed products from the 60S subunits by ANKZF1/VMS1 (11), and degradation of aberrant peptides by the Ltn1/VCP/NEMF complex (4). The biological significance of the CAT-tailed translation products generated in the RQC remains unclear. On one side of the coin, CAT-tails can push lysine residues out of the ribosome exit channel to achieve Ltn1-mediated ubiquitination (12), or promote degradation of faulty nascent peptide chains (13, 14); on the other side, CAT-tailed proteins are prone to form detergent-insoluble aggregates (15, 16). In addition, depending on the nature and subcellular localization of the original target, CAT-tailed proteins may also have unique functions, but this is currently poorly understood. Importantly, CAT-tailed proteins have been found in a variety of human neurodegenerative diseases and are believed to play important roles in their pathogenesis (17-19).

The increased abnormalities in translation have been found in cancer cells, such as stop codon readthrough (20), frame-shifting (21), and oxidative stress-induced ribosome stalling (22), indicating the possible involvement of the RQC pathway. However, despite the detection of CAT-tail modification on mitochondrial proteins caused by impaired RQC in HeLa cells (17), there is still a lack of mechanistic studies on the role of RQC factors in cancer biology. The expression of many RQC factors (e.g., ASCC3, ABCE1, ANKZF1 and VCP) has been shown to be dysregulated in cancer cells (23-26). Intriguingly, different RQC factors, or even the same factor, may exhibit completely opposite functions in oncogenesis and tumor suppression under distinct conditions. Inhibition of ABCE1, ASCC3 and VCP inhibits cancer cell growth and survival (23, 24, 26), whereas inhibition of NEMF/Clbn and ZNF598 may promote cancer growth and survival (27, 28). This suggests that the role of RQC factors in cancer cells is sophisticated and highly genetic and environmental context dependent. A recent study investigated the mechanism of ANKZF1 in mitochondrial proteostasis and its impact on the malignant progression of GBM (29). However, the nonphysiologically mitochondria-targeted GFP was used in the study to induce the matrix proteotoxicity, and the role of endogenous mitochondrial proteins in this process remains unclear.

The co-translational import defects triggered by mitochondrial stress induce CAT-tailing not only on the mitochondrial C-I30 protein (complex-I 30 kD subunit protein, also known as NDUFS3), but also widely on other nuclear genome-encoded mitochondrial proteins (17, 30). How these mitochondrial stress-induced CAT-tailed (msiCAT-tail) proteins participate in and influence the core of mitochondrial biology has not yet been studied. Since CAT-tailing imparts new properties to target proteins, it is likely to contribute to some unique characteristics of cancer cell mitochondria, such as mitochondrial hyperpolarization (31, 32) and high mitochondrial membrane potential-related resistance to drug-induced apoptosis (33-35). Mitochondrial membrane potential is formed by the proton (H⁺) concentration gradient across the mitochondrial inner membrane. It is generated and maintained by oxidative phosphorylation (OXPHOS), in which H⁺ ions are shuttled through the electron transport chain composed of four complexes (I to IV), resulting in the accumulation of H⁺ in the intermembrane space (36). ATP synthase (complex-V) then harnesses the energy from the H⁺ gradient by promoting its influx into the mitochondrial matrix and produces ATPs in the process (37). Despite increased metabolic demands, many cancer cells have reduced OXPHOS (38). It remains unclear how cancer cells maintain or even increase mitochondrial membrane potential in the absence of OXPHOS (31).

In this study, the inventors focused on msiCAT-tailing modification on the mitochondrial ATP synthase F1 subunit alpha (ATP5α). The inventors observed that msiCAT-tailed ATP5α is present in GBM. The short-tailed ATP5α can incorporate into the ATP synthase, increase mitochondrial membrane potential, and inhibit the assembly and opening of mitochondrial permeability transition pore (MPTP). Therefore, the presence of msiCAT-tailed ATP5α enhances the resistance of GBM cells to staurosporine (STS)- and temozolomide (TMZ)-induced programmed cell death and promotes cancer cell survival, proliferation, and migration. Conversely, inhibition of msiCAT-tailing suppresses cancer cell growth and resensitizes GBM cells to apoptosis. Our study elucidates the role of CAT-tailed mitochondrial proteins in cancer cells, reveals the importance of the RQC pathway in cancer biology, and indicates that components and products in the RQC pathway may be effective therapeutic targets for GBM.

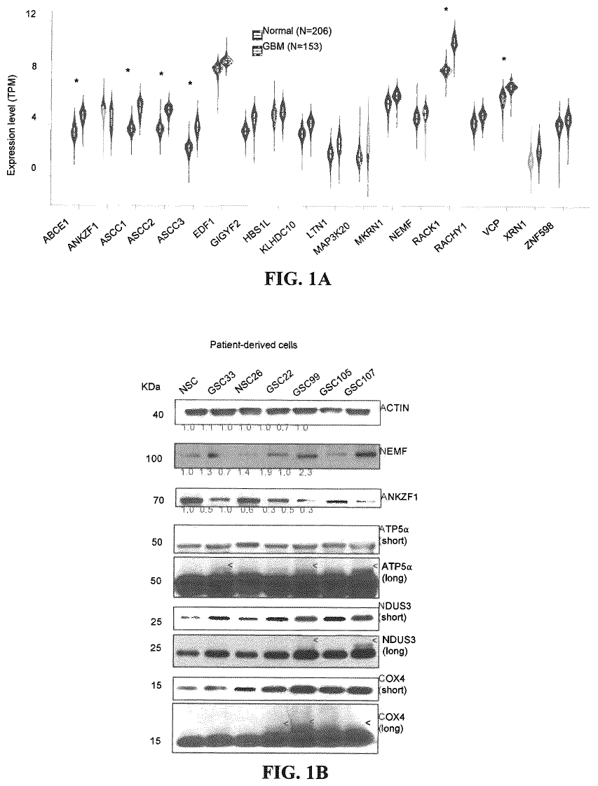

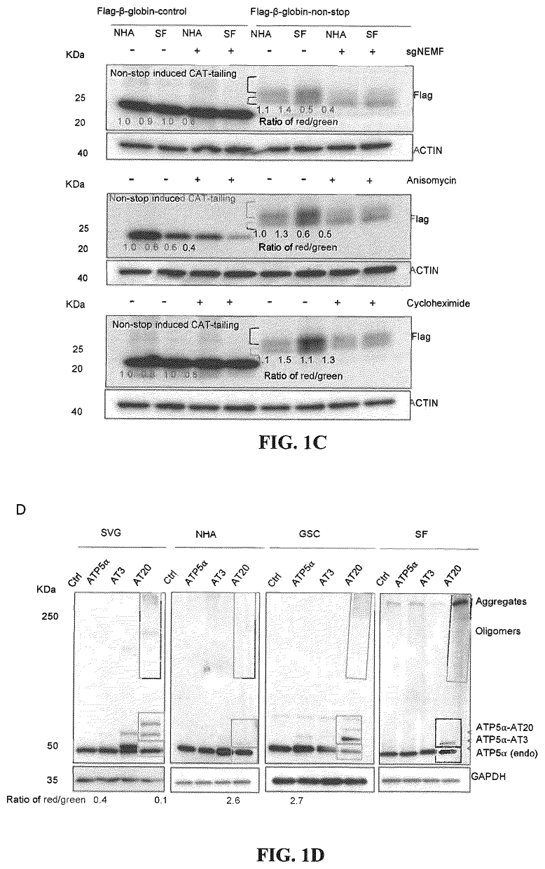

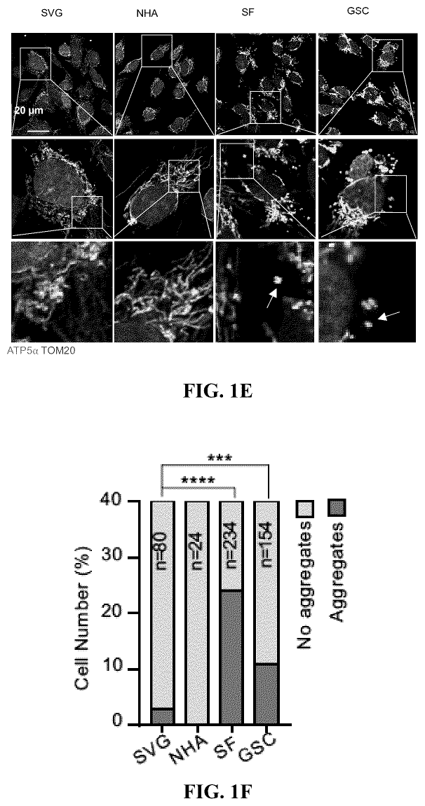

Presence of msiCAT-tailed Proteins in Glioblastoma Cells. While dysregulation of individual ribosome-associated quality control (RQC) factors is documented across various cancers (e.g., adenocarcinoma, non-small cell lung, prostate, and colon carcinomas), a comprehensive analysis of the RQC pathway in glioblastoma (GBM) has been lacking (23-26). The inventors' analysis of transcriptomic data from a cohort of 153 GBM patients and 206 healthy controls, sourced from public datasets, revealed significantly elevated expression (log FC (fold change) >1; adj.P.Val <0.001) of RQC pathway genes, such as ABCE1, ASCC1-3, RACK1, and VCP, in GBM cells. Conversely, ANKZF1 was significantly downregulated (log FC=−0.43, adj.P.Val=0.0005) (FIG. 1A). The expression change in these genes suggests RQC pathway activation and potential accumulation of CAT-tailed proteins in GBM. Mitochondrial stress-induced protein mitochondrial Complex-I 30 kDa (C-I 30, also known as NDUS3), an endogenous RQC substrate with msiCAT-tails, was previously identified in HeLa cells (17). Examination of patient-derived GBM stem cells (GSCs) and normal neural stem cells (NSCs) revealed that GSCs, unlike NSCs, exhibited several msiCAT-tailed mitochondrial proteins, including NDUS3, COX4 (Cytochrome c Oxidase subunit 4), and ATP5α (ATP synthase F1 subunit alpha). Consistent with the detection of these msiCAT-tailing signals, increased NEMF (Nuclear Export Mediator Factor) levels (10) and decreased ANKZF1 (Ankyrin Repeat and Zinc-finger Peptidyl tRNA Hydrolase 1) expression (11) were observed in patient-derived GSCs (FIG. 1B), further indicative of enhanced CAT-tailing activation, mirroring bioinformatics findings in GBM samples. A murine GBM model exhibited analogous RQC pathway alterations, with increased NEMF and decreased ANKZF1 expression in transplanted SB28 gliomas compared to normal brain tissue.

The subsequent experiments were conducted using two GBM cell lines, SF268 (SF in figures) (40) and GSC827 (GSC in figures) (41), and two control cell lines, SVG p12 (SVG in figures) and Normal Human Astrocytes E6/E7/hTERT (NHA in figures) (42). RQC protein expression analysis revealed decreased ANKZF1 and increased ABCE1, ASCC3, and NEMF expression in GSC827 and SF268 cells, consistent with findings in patient-derived GSCs. Intriguingly, induction of CAT-tailing on a Flag-tagged β-globin reporter via a non-stop protein translation system demonstrated significantly higher CAT-tailed protein (0-globin-nonstop) production in GBM cells (43). This process was inhibitable by the CAT-tailing elongation inhibitor anisomycin and NEMF knockdown (sgNEMF), but not cycloheximide treatment, as evidenced by a decreased ratio of CAT-tailed (top bracket) to non-CAT-tailed bands (bottom bracket) (FIG. 1C).

Next, to investigate the biological implications of CAT-tailing, artificial CAT-tails were introduced to mitochondrial proteins. Due to the variability in CAT-tailing, prior research simulated this process by adding alanine-threonine (AT) repeat tails to the C-terminus of mitochondrial proteins (17). ATP5α, a highly abundant mitochondrial protein with roles in cancer, was selected to study the unique functions of CAT-tailed forms (45, 46). siATP5α knockdown first confirmed the upper band signal in GSCs as authentic ATP5α, demonstrated by its disappearance concurrent with the main band's weakening. Then, the inventors confirmed that this upper band signal corresponded to changes in CAT-tailing, which could be effectively inhibited by NEMF knockdown and anisomycin treatment. Due to the indistinct nature of the endogenous msiCAT-tailed ATP5α signal, exogenously expressed Flag-ATP5α was utilized here.

To investigate the potential new function provided by CAT-tailed proteins, control (SVG and NHA) and GBM (SF268 and GSC827) cell lines overexpressed ATP5α with three (ATP5α-AT3) or twenty (ATP5α-AT20) AT repeats. Consistent with earlier findings, only the long-tailed ATP5α-AT20 exhibited post-translational modifications and detergent-resistant insoluble aggregates, appearing as slower migrating bands and a high molecular weight smear in protein electrophoresis (FIG. 1D). Based on comparing exogenously expressed (indicated by top two boxes) to endogenous proteins (indicated by lowest boxes), GBM cell lines (GSC827, SF268) showed increased accumulation of ATP5α-AT20 compared to control cells (SVG, NHA). This accumulation may occur due to increased stability and reduced degradation of long-tailed proteins, a malfunctioning protein quality control system, enhanced cellular tolerance to protein accumulation, or a combination of these factors. Subcellular localization analysis showed that the short AT tail (AT3) did not significantly alter ATP5α's mitochondrial localization, similar to the tailless protein. However, a significant portion of ATP5α-AT20 was found in the cytoplasm near mitochondria, forming protein aggregates, with the highest proportion in highly malignant GSC cells. Notably, poly-glycine-serine tails (short, GS3, and long, GS20) did not induce insoluble protein aggregation or intracellular punctate distribution, highlighting the importance of specific amino acid composition.

Importantly, in GBM cells, both exogenous tailed proteins and the endogenous ATP5α formed clusters attached to the outer mitochondrial membrane (FIGS. 1E, 1F). Similar aggregate formation in GBM cells was also observed with other mitochondrial proteins, such as NDUS3. Furthermore, the inventors examined the mouse GBM models. Akin to in vitro culture, ATP5α in transplanted SB28 glioma formed more punctate signals and did not always colocalize with the mitochondrial marker TOM20. These findings collectively indicate a disruption of the RQC pathway, leading to the presence of msiCAT-tailed proteins in GBM cells.

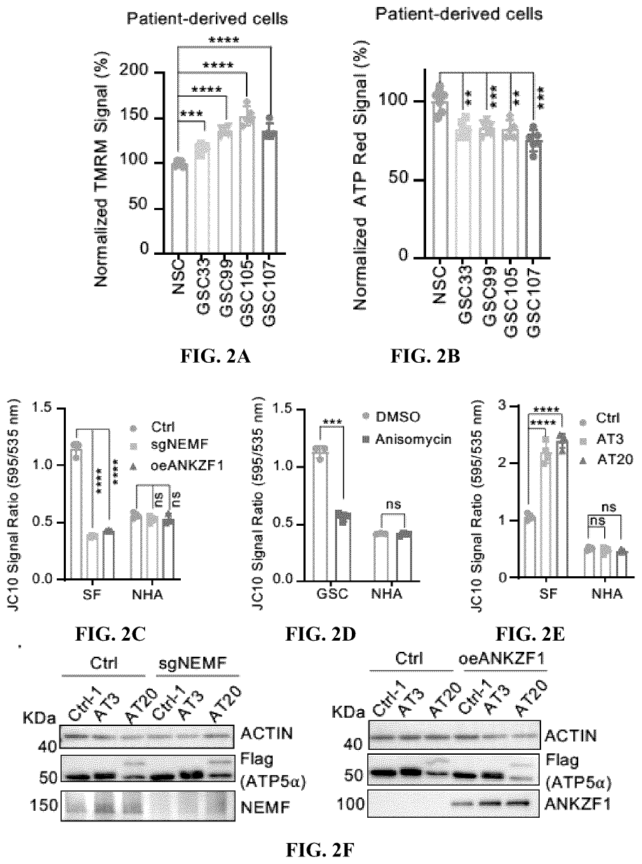

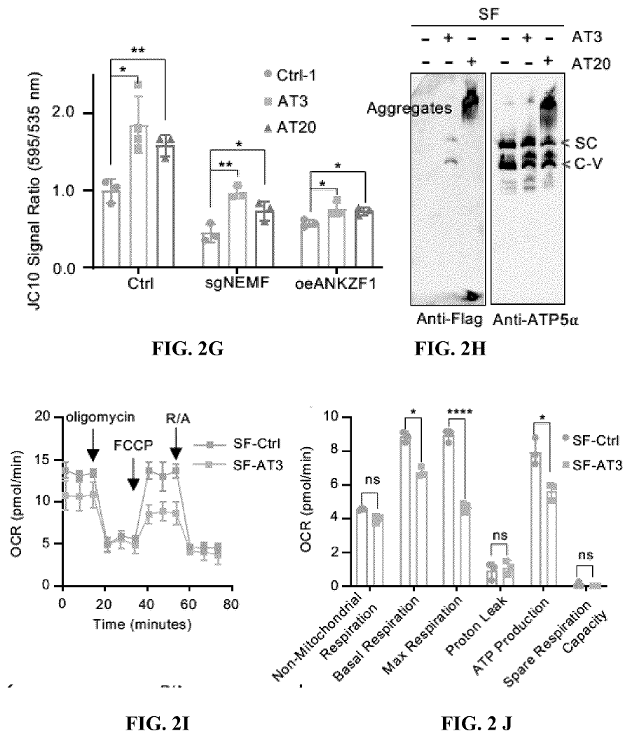

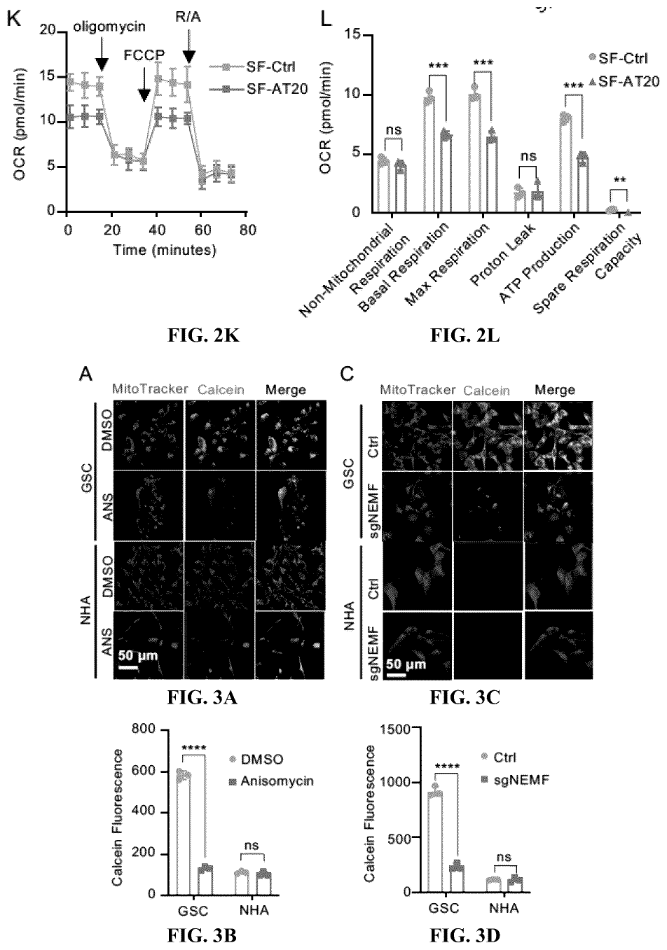

msiCAT-tailed ATP5α Elevates Mitochondrial Membrane Potential (ΔΨm). Some cancer cells exhibit altered mitochondrial physiology, maintaining or increasing mitochondrial membrane potential (ΔΨm) despite reduced respiration. This was observed in patient-derived GSC cells, which demonstrated higher ΔΨm but lower ATP production than control NSC cells (FIGS. 2A, 2B).

Similarly, GBM cell lines, GSC827 and SF268, displayed comparable or higher ΔΨm and lower ATP levels relative to the control NHA cell line (42). Genetic inhibition of msiCAT-tailing, via NEMF knockdown (sgNEMF) or ANZKF1 overexpression (oeANZKF1), as well as pharmacological inhibition by anisomycin treatment, effectively reduced ΔΨm in GBM cells but not in NHA cells (FIGS. 2C, 2D).

Next, the inventors show the impact msiCAT tail proteins on mitochondrial function. Expression of Flag-tagged ATP5α-AT3 and ATP5α-AT20 in GBM and control cell lines elevated ΔΨm specifically in GBM cells (FIG. 2E). Overexpression of ATP5α-GS3 and ATP5α-GS20 did not exhibit this effect. Surprisingly, even with suppressed endogenous CAT-tailing through sgNEMF and oeANZKF1 in GSC cells, the introduced AT3 and AT20 proteins could still effectively elevate ΔΨm (FIGS. 2F, 2G). This finding suggests that CAT-tailing of ATP5α may be a significant contributor to the observed mitochondrial phenotype (FIG. 2G). Blue Native Polyacrylamide Gel Electrophoresis (BN-PAGE) illustrated distinct effects based on CAT-tail length. ATP5α-AT3 integrated into the mitochondrial respiratory chain complex, whereas ATP5α-AT20 formed high molecular weight complexes or remained as monomers (FIG. 2H). In mitochondrial physiological activity assays using the Agilent Cell Mitochondrial Stress Test, the oxygen consumption rate (OCR) was directly measured to assess mitochondrial respiration. These findings demonstrate that expressing both ATP5α-AT3 and ATP5α-AT20 negatively impacted mitochondrial oxidative phosphorylation. This impairment leads to a reduction in ATP synthesis, basal respiration, and maximal respiration rates (FIGS. 2I-2L). These data suggest that both short and long tails on ATP5α proteins influence mitochondrial function, although potentially through different mechanisms. Short CAT-tails may directly act on ATP synthase function and thus affect the respiratory chain complex, while long CAT-tails form protein aggregates, causing mitochondrial proteostasis stress and thus indirectly affecting mitochondrial respiration (17, 29). This differential impact of CAT-tail length suggests a nuanced regulation of mitochondrial function mediated by ATP5α modifications.

msiCAT-tailing Influences Mitochondrial Permeability Transition Pore (MPTP) Dynamics. Beyond its traditionally recognized role in ATP production, the F₁F₀ ATP synthase has garnered increasing attention as a potential structural component of the mitochondrial permeability transition pore (MPTP) complex (47-49). Given the possibility that CAT-tailed proteins like ATP5α might modulate MPTP function, this investigation sought to elucidate the mechanism by which msiCAT-tailing modulates MPTP dynamics (open-close state). Comparative analyses conducted in GBM and control cells revealed that MPTP in GSC827 cells predominantly exists in a closed conformation, indicated by strong Calcein signals. Notably, the treatment of anisomycin, a pharmacological CAT-tailing inhibitor, effectively induced MPTP opening in GSC827 cells, as indicated by decreased Calcein signals (FIGS. 3A, 3B). This effect was concomitant with the diminished aggregation of endogenous ATP5α (FIGS. 3E, 3F). Furthermore, corroborative evidence was obtained through genetic manipulation. Specifically, genetic inhibition of CAT-tailing via NEMF knockdown (sgNEMF) resulted in a similar decrease in Calcein signaling and a reduction in ATP5α accumulation (FIGS. 3C, 3D, 3G, 3H), aligning with the results obtained using anisomycin. In contrast, treatment with cycloheximide, a general translation inhibitor, did not significantly alter Calcein or ATP5α aggregation signals, showing that non-specific translation inhibition does not impact the mitochondrial MPTP state. The crucial role of CAT-tail modifications on ATP5α in modulating MPTP status was further substantiated by the observation that overexpression of artificially synthesized AT repeat tails (AT3 and AT20) restored Calcein signals despite the inhibition of endogenous CAT-tailing.

The MPTP is recognized to participate in the transient efflux of protons, calcium ions (Ca²⁺), and other signaling molecules from the mitochondrial matrix during brief opening episodes (50). To quantitatively evaluate the MPTP open/closed state, the mitochondrial Ca²⁺ Retention Capacity (CRC) assay was employed, which measures the amount of Ca²⁺ required to elicit MPTP opening. These results revealed that GSC827 cells exhibited a greater CRC than NHA cells. Pre-treatment with anisomycin or knockdown of NEMF (sgNEMF) significantly decreased the CRC in GBM cells, indicating MPTP opening upon the loss of CAT-tailed proteins (FIGS. 3I, 3J). Consistent with Calcein staining results, cycloheximide treatment did not substantially alter CRC measurements. Conversely, enhancing CAT-tailing (e.g., via oeNEMF and siANKZF1) led to an increase in CRC, although this effect was less pronounced in GSCs, potentially due to their inherently active CAT-tailing.

To further investigate the impact of specific AT repeat tails on MPTP opening, artificial AT repeat tails on ATP5α were introduced into GBM cells. It was found that the short AT tail (AT3) inhibited MPTP opening, while the long AT tail (AT20) displayed a weaker effect (FIGS. 3K, 3L), potentially due to their different integration into ATP synthase (FIG. 2G). Complex co-immunoprecipitation assay did not detect direct interactions between ATP5α with AT3 or AT20 tails and MPTP components Cyclophilin D (Cyp-D) and adenine nucleotide translocator 2 (ANT2). However, Cyp-D expression was reduced upon ectopic expression of ATP5α-AT3 and ATP5α-AT20, suggesting decreased MPTP formation. Intriguingly, BN-PAGE analysis revealed that both ATP5α-AT3 and ATP5α-AT20 altered ANT1/2-containing complexes, with expected bands disappearing (indicated by *) and aggregates forming (at the top), supporting the notion that ATP synthase is integrated into the MPTP supercomplex due to the spatial proximity of the ANT1/2 complex and ATP synthase (FIG. 3M). In conclusion, msiCAT-tailed ATP5α proteins, particularly those with short AT3 tails, are integrated into ATP synthase and have a substantial influence on modulating MPTP status.

msiCAT-Tailing Boosts GBM Cell Migration and Resistance to Apoptosis. The elevated mitochondrial membrane potential (ΔΨm) and constricted MPTP resulting from msiCAT-tailed ATP5α and other mitochondrial proteins may enhance cellular stress resilience. The inventors first investigated how the msiCAT-tailing mechanism affects GBM cells at the cellular level. MTT assays (51) revealed that overexpressing short (AT3) and long (AT20) AT repeat tails, fused to ATP5α, significantly improved GBM cell viability, but not that of NHA cells (FIGS. 4A, 4B). However, short (GS3) and long (GS20) GS repeat tails did not affect GBM cell viability. In addition, in vitro transwell migration assays (52) and wound healing assays (53) showed that GBM cells overexpressing AT repeat-tailed ATP5α exhibited increased cell invasion and accelerated wound healing, indicating enhanced cell migration (FIGS. 4C, 4D, 4F). Notably, neither ATP5α alone nor GS repeat-tailed proteins showed comparable changes (FIG. 4E). Furthermore, overexpressing AT3- and AT20-tailed proteins effectively conferred phenotypes associated with increased GBM cell activity, such as enhanced survival and migration, even with inhibited endogenous CAT-tailing machinery activity (e.g., sgNEMF and oeANKZF1) (FIGS. 4E-4G). It is worth noting that ANKZF1 knockdown in U87 and U251 cell lines can cause aberrant mitoGFP accumulation, possibly reducing cellular adaptability (29), suggesting varying mitochondrial adaptability to proteostasis stress across cell lines. Supporting this, initial experiments showed that mild expression of ATP5α-AT3 and ATP5α-AT20 did not induce strong mitochondrial proteotoxic responses, as evidenced by the lack of significant upregulation in LONP1, mtHSP70, and HSP60 mRNA levels.

GBM cells exhibit increased resistance to staurosporine (STS)-induced apoptosis, supported by fewer TUNEL-positive cells and markedly diminished PARP-1 (Poly ADP-ribose polymerase) cleavage, a marker of AIF-mediated apoptosis (54). To investigate the role of CAT-tailed ATP5α proteins in this resistance, the inventors overexpressed proteins with mimetic tails in GBM cells. Overexpression of both short tail (ATP5α-AT3) and long tail (ATP5α-AT20) significantly enhanced resistance to STS-induced apoptosis, as shown by TUNEL staining (FIGS. 4H, 4I) and flow cytometry, indicating a strong link between protein CAT-tailing and tumorigenesis. In contrast, control short (GS3) and long (GS20) GS tails failed to confer such resistance. Consistent with these findings, overexpression of artificial CAT-tailed ATP5α proteins also increased the resistance of GBM cells to temozolomide (TMZ)-induced apoptosis (FIG. 4J). Taken together, these results suggest that RQC-induced CAT-tailing on ATP5α protein plays a role in GBM resistance to drug-induced apoptosis.

GBM Cell Progression is Hindered by RQC Pathway Inhibition. Prior research indicates the RQC pathway's mediated msiCAT-tailing plays an important role in GBM progression, suggesting it as a potential therapeutic target. To explore this, patient-derived Glioblastoma Stem Cell (GSC) lines were treated with anisomycin, the inhibitor of CAT-tailing. GSC lines displayed higher sensitivity to anisomycin than normal neural stem cells (NSCs) (FIG. 5A). Similarly, genetic inhibition of the RQC pathway via NEMF knockdown (sgNEMF) or ANKZF1 overexpression (oeANZKF1) in the SF268 GBM cell line also suppressed GBM growth (FIG. 5B). Notably, control NHA cell proliferation was also inhibited by these genetic changes, indicating the broad significance of NEMF and ANKZF1 in cell proliferation (FIG. 5C). The RQC pathway appears to have a more pronounced effect on GBM cell migration. In in vitro transwell assays, sgNEMF or oeANZKF1 notably decreased GBM cell migration without affecting NHA cells (FIGS. 5D, 5E). Consistently, anisomycin treatment impaired GSC cell migration, but not NHA cell migration (FIGS. 5F, 5G).

Further investigation revealed the RQC pathway's involvement in GBM cell anti-apoptosis, with initial findings pointing to alterations in mitochondrial functions. Prior studies demonstrated that genetic or pharmacological inhibition of the RQC pathway led to a significant decrease in GBM mitochondrial membrane potential (ΔΨm) (FIGS. 2C, 2D). In GSC cells, anisomycin treatment promoted mitochondrial permeability transition pore (MPTP) opening, an effect not seen in NHA cells (FIGS. 3A-3D). Consequently, GBM cell lines with genetically or pharmacologically inhibited RQC pathways were more susceptible to STS-induced apoptosis, evidenced by elevated executioner caspase 3/7 activity, enhanced PARP-1 cleavage, increased TUNEL staining (FIGS. 5H-5K), and flow cytometry analysis. Notably, general translation inhibition using cycloheximide did not elicit the same apoptotic response. Finally, the RQC pathway was also implicated in temozolomide (TMZ)-induced cell death. Combining anisomycin with TMZ significantly reduced GBM cell survival (FIG. 5L) and effectively inhibited GSC spheroid growth (FIGS. 5M, 5N). In summary, the RQC pathway plays a critical role in multiple aspects of GBM progression, including proliferation, migration, and survival under apoptotic stress.

This study explored the role of RQC and its products, especially the mitochondrial proteins modified by the msiCAT-tailing mechanism, in cancer cells. Glioblastoma (GBM) cells were used as a model to conduct the studies, but this does not mean that these phenomena only occur in GBMs. They may be widely present in the occurrence and development of cancer. Here, the inventors focused on msiCAT-tail modifications on the mitochondrial protein ATP5α. In this model, due to the specific function of mitochondrial ATP synthase itself, msiCAT tail-modified proteins endow GBM cells with several unique properties, such as maintaining or increasing mitochondrial membrane potential despite reducing ATP production, promoting the survival and migration of GBM cells, and providing resistance to STS-induced apoptosis, and this last point may be related to regulating the open-close state of MPTP (FIG. 6). These characteristics promote the malignancy of tumor cells, thus inhibiting the RQC pathway may be used as an effective adjuvant treatment to complement existing chemotherapy methods. In addition, studying the behavior of ATP synthase in cancer is also of special significance. In carcinogenesis, ATP synthase is often found re-localized to the plasma membrane and is known as the ectopic ATP synthase complex (eATP synthase). These eATP synthases have catalytic activity and can promote the generation of ATP in the extracellular environment to establish a suitable tumor microenvironment (55). eATP synthase was shown to be first assembled in mitochondria and then delivered to the cell surface via microtubules (46). However, what kind of ATP synthase can be delivered to the plasma membrane is not clear. Studying the localization of CAT-tailed eATP synthase in the future may provide us with clues.

It is worth noting that more than one mitochondrial protein can be CAT-tailed in cancer cells. It is very likely that the multiple nuclear-genome encoded mitochondrial proteins have been CAT-tailed in the similar way, and due to the different properties of these base proteins, msiCAT-tailed peptides may have diverse effects on mitochondria or cells. For example, CAT-tailed COX4 protein may contribute to a more significant and direct reduction in mitochondrial respiratory efficacy. It may be interesting to investigate their roles separately, as the combined effects of the individual defects may be key to helping understand the observed mitochondrial changes in cancer. Indeed, in a recent study, the authors found that knocking down ANKZF1 inhibited the progression of GBM by causing the accumulation of abnormal proteins within mitochondria (29). Combined with the data herein, the inventors conclude that the balance of ANKZF1 expression and activity is essential for cancer proliferation. Excess or deficiency may cause changes in cell adaptability. A minor drawback is that in their study, the authors used a mitochondrial-localized non-stopped GFP protein to induce proteostasis stress, and there was no direct biochemical evidence for detecting the CAT-tailed proteins. Here, the focus was on endogenous proteins to analyze the impact of target proteins on mitochondria in more detail. The consideration is that abundantly expressed non-physiological ectopic proteins may cause general proteostasis failure and thus mask the specific functions of endogenous proteins. In addition, the cell lines used in the two studies were different. GSC is a patient-derived GBM cell line with more stemness, and its mitochondrial status and RQC pathway activity may be different from those of the U87 or U251 cell lines. Therefore, rather than thinking that the conclusions from the two studies are in conflict, it is better to say that they are complementary, and both prove the importance of RQC in tumorigenesis. Our study explores the mechanistic role of RQC pathway in GBM and provides new potential targets for future treatments.

Comprehensive analysis of how many nuclear genome-encoded mitochondrial proteins are modified through the msiCAT-tailing mechanism using advanced mass spectrometry is an interesting topic and deserves further study. In a recent study published in Cell Reports, Lv and colleagues found that the cytoplasmic E3 ligase Pirh2 and the mitochondrial protease ClpXP can complement the classic NEMF-ANKZF1 to degrade mitochondrial protein aggregation caused by ribosome stalling (56). The elevation of ClpXP in various cancers (57) may be explained by increased msiCAT-tailing products in mitochondria, and the impact of ClpXP on mitochondrial RQC also depends on future research. ClpXP also regulates the levels of a variety of mitochondrial proteins. In these experiments, it was found that ATP5α proteins without msiCAT-tails are most difficult to ectopically express; short tails (AT3) containing proteins are better, while proteins with long tails (AT20) have higher expression levels and form SDS-insoluble protein aggregates. This regulation may also be achieved through the degradation of ClpXP. Another possibility is regulation at the transcriptional level. The peroxisome proliferator-activated receptor gamma co-activator (PGC-1α) is a master regulator of mammalian mitochondrial biogenesis (58). P GC-1α can bind and activate nuclear transcription factors, leading to the transcription of nuclear genome-encoded mitochondrial proteins and the mitochondrial transcription factor Tfam. Tfam subsequently activates transcription and replication of the mitochondrial genome (59). A careful examination of the mRNA levels of the msiCAT-tailed targets and the binding of PCG1α and Tfam to transcriptional elements can be used to distinguish between these two possibilities.

MPTP is an ion nonselective, calcium-dependent, and multifunctional supramolecular channel that penetrates both the inner membranes of mitochondria. Although the function and regulation of MPTP have been widely studied, an understanding of its molecular structure has remained unclear (60). Currently, multiple structural models of MPTP have been proposed: (i) the VDAC (voltage-dependent anion channel)/ANT (adenine nucleotide translocator)/Cyp-D (cyclophilin D) model (61). However, subsequent genetic studies have shown that whether these proposed proteins are structural components of MPTP is still highly controversial (62-65); and (ii) the ATP synthase model of MPTP. In this model, dimers of ATP synthase or reconstitution of ATP synthase (c-ring) may form the molecular structure of MPTP (47, 48). By way of explanation, and not a limitation of the present invention, this hypothesis is intriguing and the role of ATP synthase as a component of the pore structure has been the subject of many conflicting studies yet has not yet been unequivocally confirmed. iii) the current prevalent hypothesis is that ANT and ATP synthase together form a large complex (ATP synthasome), and Cyp-D regulates the dynamics of ATP synthasome (66).

MPTP is regulated by the physiological voltage of mitochondrial membrane potential (67, 68), and conversely, the opening of MPTP can also impact ion homeostasis and energy metabolism in the mitochondrial matrix. In this study, two threads converge here. The msiCAT-tailed ATP5α protein helps cancer cells: (i) maintain a high/stable membrane potential, which can desensitize MPTP induction, and (ii) directly participate in MPTP assembly, thus inhibiting MPTP's function. Intriguingly, MPTP is an important mediator of cell death. However, for a long time, no reports have been published to clearly confirm or refute the hypothesis, that is more closed (inhibition) MPTP could be an important feature of cancer cells, which can help them escape drug-induced programmed cell death. Here the inventors present evidence supporting showing that msiCAT-tailed ATP5α protein helps cancer cells: (i) maintain a high/stable membrane potential, which can desensitize MPTP induction, and (ii) directly participate in MPTP assembly, thus inhibiting MPTP's function. The inventors observed that GBM cells, especially GSC cells, have a more closed MPTP than control cells. A more closed MPTP is directly related to the CAT-tailing modification of the ATP synthase subunit. This is also consistent with evidence that genetic mutations or post-translational modifications in certain ATP synthase subunits can affect MPTP activity (69, 70).

Cell lines and cell culture conditions. The human astroglia cell line SVG p12 (ATCC, cat. CRL-8621) and the human glioma cell line SF268 are provide by Dr. Rongze Olivia Lu. Both cell lines were cultured in DMEM (ATCC, cat. #302002) with 10% FBS (Biowest, cat. 51620-100) and penicillin/streptomycin (Gibco™, cat. 15140122). SF268 clones should be maintained in complete DMEM supplemented with 400 μg/mL G418 (Gibco, cat. 10131027). The 0.25% trypsin solution (ATCC, cat. #SM2003C) were used to passage cells. The normal human astrocytes NHA E6/E7/hTERT cell line was from Dr. Russell O. Pieper, UCSF Brain Tumor Research Center. Cells are cultured in ABM™ Basal Medium (Lonza, cat. CC-3187) and AGM™ SingleQuots™ Supplements (Lonza, cat. CC-4123). Corning® Accutase™ Cell Detachment Solution (Corning, cat. 25058CI) were used to passage cells. GSC827 cells, a patient-derived human glioma stem cell line, was from Dr. Chun-Zhang Yang at NIH. The NSC, NSC26, patient-derived GSC33, GSC22, GSC99, GSC105 and GSC107 cell lines used in this study were kindly provided by Dr. John S Kuo at the University of Texas, Austin. GSC cells were cultured in Neural basal-A Medium (Gibco, cat. #10888022) with 2% B27 (Gibco, cat. #17504044), 1% N2 (Gibco, cat. #17502048), 20 ng/ml of EGF and FGF (Shenandoah Biotechnology Inc. cat. PB-500-017), Antibiotic-Antimycotic (Gibco, cat. #15240062) and L-Glutamine (Gibco, cat. #250300810). Cells could be cultured in both spherical and attached (on Geltrex, Thermo Fisher, cat. A1413202) forms. Corning® Accutase™ Cell Detachment Solution (Cornin, cat. 25058CI) were used to passage cells.

Cells were transfected with X-tremeGENE™ HP DNA Transfection Reagent (Sigma, cat. 6366244001) following the standard protocol. For single clone selection, SF268 cells were treated with 800 μg/ml G418 for 5 days. The cells were then seeded into 96-well plate at a density of 1/100 μL. Positive clones were verified by immunofluorescence staining and immunoblotting. Cells were maintained in complete DMEM containing 400 μg/mL G418. GBM cell lines were subjected to a 4-hour pre-treatment at 37° C. using either anisomycin (20 nM or 200 nM, Fisher Scientific, cat. AAJ62964MF) or cycloheximide (100 μg/mL, Fisher Scientific, cat. AC357420010) in medium, as detailed in the conducted experiments.

Primers, Plasmids and viruses. Plasmids pcDNA3.1+/C-(K)-DYK-ATP5F1A (pATP5α control), pcDNA3.1+/C-(K)-DYK-ATP5F1A-AT3 (pATP5α-AT3), pcDNA3.1+/C-(K)-DYK-AT20 (pATP5α-AT20), pcDNA3.1+/C-(K)-DYK-ATP5F1A-GS3 (pATP5α-GS3), and pcDNA3.1+/C-(K)-ATP5F1A-DYK-GS20 (pATP5α-GS20) were generated by GenScript Inc. Plasmids pCMV-5×FLAG-β-globin-control (5FBG-Ctrl) and pCMV-5×FLAG-β-globin-non-stop (5FBG-nonstop) were generated by Dr. Hoshino (Nagoya City University) and Dr. Inada (Tohoku University) (43).

Viruses (and plasmid used to generate viruses) pLV[CRISPR]-hCas9:T2A:Neo-U6>Scramble[gRNA #1](sgControl), pLV[CRISPR]-hCas9:T2A:Neo-U6>hNEMF[gRNA #1579](sgNEMF), pLV[Exp]-Bsd-EF1A>ORF_Stuffer (pLV-control), pLV[Exp]-EGFP:T2A:Puro-EF1A>mCherry (pLV-control-2), pLV[Exp]-Bsd-EF1A>hANKZF1[NM_001042410.2]/HA (oeANKZF1), and pLV[Exp]-mCherry/Neo-EF1A>hANKZF1[NM_001042410.2](oeANKZF1) were made by VectorBuilder Inc.

Primers (5′ to 3′) used for RT-PCR are:

| | | SEQ |
|---|---|---|
| | | ID |
| Target | Sequence | NO: |
| LONP1 | lonp1_forward: TGCCTTGAACCCTCTCTAC | 1 |
| | lonp1_reverse: TCTGCTTGATCTTCTCCTCC | 2 |
| mtHSP70 | mthsp70_forward: ACTCCTCCATTTATCCGCC | 3 |
| | mthsp70_reverse: ACCTTTGCTTGTTTACCTTCC | 4 |
| HSP60 | hsp60_forward: ACCTGCTCTTGAAATTGCC | 5 |
| | hsp60_reverse: CAATCCCTCTTCTCCAAACAC | 6 |
| ACTB | actb_forward: TGTTTGAGACCTTCAACACC | 7 |
| | actb_reverse: ATGTCACGCACGATTTCC | 8 |

Neurosphere formation assay of GSCs. The GSC spheroids were dissociated using Accutase for 2 minutes. Cells were resuspended in single cell suspension and grown under non-adherent conditions. Cells were seeded in 12-well plates at a density of 0.25×10⁶ cells/well and cultured in 3 mL culture medium for 24 hours. 20 nM of anisomycin and 150 μM of temozolomide (TMZ) were added to the culture medium and treated for 96 hours. Spheroids were images under 10× objective, captured using QCapture, and analyzed with imageJ. Spheroids larger than 50 μm were counted.

Differential gene expression analysis using the public database. The raw RNA-seq data used to perform the analysis were obtained from the University of California Santa Cruz Xenabrowser (cohort: TCGA TARGET GTEx, dataset ID: TcgaTargetGtex_rsem_gene_tpm, https://xena.ucsc.edu/), and then subsets included only TCGA glioma (GBM), GTEx Brain Frontal Cortex, and GTEx Cortex samples. Differential expression analysis was conducted using the “limma” package (R version: 4.3.1). The benjamini-Horchberg method was used for multiple testing correction to control the false discovery rate (FDR). Cut-off of adjusted p-value (adj.P.Val) was set at 0.001; cut-off of the absolute fold change was set at 2 (log FC>1).

Immunostaining. Cells were cultured on sterile coverslips until 80% confluency. For immunostaining, cells were washed with phosphate buffered saline (PBS) solution thrice. Then, 4% formaldehyde (Thermo Fisher, cat. BP531-500) was applied to cells for fixation for 30 minutes at room temperature. After fixation, cells were washed with PBS solution containing 0.25% TritonX-100 (PBSTx) (Thermo Fisher, cat. T9284) thrice, and blocked with 5% normal goat serum (Jackson Immuno, cat. 005-000-121) for 1 hour at room temperature. Cells were then incubated with primary antibodies overnight in a humidified chamber at 4° C. The next day, cells were washed by PBSTx thrice and incubated with secondary antibodies for 2 hours at room temperature. After washing, cells were stained with 300 nM DAPI (Thermo Fisher, cat. 57-481-0) for 5 minutes at room temperature and mounted in Fluoromount-G Anti-Fade solution (Southernbiotech, cat. 0100-35). Images were taken using a Zeiss LSM 800 confocal microscope, 40× oil objective lens and Airyscan processing. The primary antibodies used in the study were rabbit anti-ATP5α (Cell Signaling, cat. #18023), mouse anti-TOMM20 (1:500, Santa Cruz, cat. 18023S), rabbit anti-MTCO2 (1:500, Abclonal, cat. sc-17764). The secondary antibodies were Alexa fluor 633-, 594-, 488-conjugated secondary antibodies (1: 300, Invitrogen, cat. A21071, A11036, A32732).

SDS-PAGE and immunoblotting. Cells or isolated mitochondria were solubilized in cell lysis buffer containing 50 mM Tris-HCl pH 7.4, 150 mM NaCl, 10% glycerol, 1% TritonX-100, 5 mM EDTA and 1× protease inhibitor (Bimake, cat. B14002). Protein concentration was measured by using the Bradford assay (BioVision, cat. K813-5000-1). Samples were separated in 4-12% Tris-Glycine gel (Invitrogen, cat. WXP41220BOX) and proteins were transferred to PVDF membrane (Millipore, cat. ISEQ00010). The membranes were then blocked with 5% non-fat dry milk (Kroger) for 50 minutes at room temperature and probed with primary antibodies overnight at 4° C. Membranes were washed with Tris buffered saline with 0.1% Tween 20 (TBST) solution thrice and then incubated with secondary antibodies for 1 hour at room temperature. Blots were detected with ECL solution (PerkinElmer, cat. NEL122001EA) and imaged by Chemidoc system (BioRad). The intensity of blots was further analyzed by ImageJ software. The primary antibodies used were mouse anti-Actin (1:1000, Santa Cruz, cat. sc-47778), rabbit anti-NEMF (1:1000, Proteintech, cat. 11840-1-AP), mouse anti-ANKZF1 (1:1000, Santa Cruz, cat. sc-398713), mouse anti-ATP5α (Abcam, cat. ab14748), mouse anti-C-130 (1:1000, Abcam, cat. ab14711), rabbit anti-COX4 (Abcam, cat. ab209727), mouse anti-Flag (1:1000, Sigma, cat. F1804), rabbit anti-ANT1/2 (1:1000, Proteintech, cat. xxx), rabbit anti-CypD (1:1000, Proteinetch, cat. 15997-1-AP). The secondary antibodies used were goat anti-rabbit IgG (1: 5000, Invitrogen, cat. G21234), goat anti-mouse IgG (1:5000, Invitrogen, cat. PI31430).

Mitochondrial isolation, Blue Native PAGE and Western blotting. Cells were homogenized using Dounce homogenizer in ice-cold homogenization buffer containing 210 mM mannitol (Fisher Sci, cat. AA3334236), 70 mM sucrose (Fisher Sci, cat. AA36508A1), 5 mM HEPES (Fisher Sci, cat. 15630106), pH 7.12, 1 mM EGTA (Fisher Sci, cat. 28-071-G), and 1× protease inhibitor. The homogenate was centrifuged at 1500 g for 5 minutes. The resultant supernatant was centrifuged at 13000 g for 17 minutes. The supernatant was collected as the cytosol portion and the pellet as mitochondria portion was washed with homogenization buffer and centrifuged at 13000 g for 10 minutes. For Blue Native PAGE, the mitochondria samples were solubilized in 5% digitonin (Thermo Fisher, cat. BN2006) on ice for 30 minutes and then centrifuged at 20000 g for 30 minutes. The supernatant contains solubilized mitochondrial proteins and mixed with 5% G-250 (GoldBio, cat. C-460-5) and 1× NativePAGE sample buffer (Invitrogen, cat. BN2008) (final G-250 concentration is 25% of the digitonin concentration). Mitochondrial protein concentration was measured by using Bradford assay. Samples were separated in 3-12% Bis-Tris Native gel (Invitrogen, cat. BN1001BOX) and then transferred to PVDF membrane. Membranes were fixed with 8% acetic acid (Thermo Fisher, cat. 9526-33), and then blocked and probed with antibodies as described above for Western blotting.

Mitochondrial membrane potential assays. Mitochondrial membrane potential of GSC cells was measured using Image-iT™ TMRM (Invitrogen, cat. I34361). Cells were cultured in 96-well black plate at a density of 1×10⁵ cells per well overnight in incubator with 5% CO₂ at 37° C. Cells were incubated with TMRM (100 nM) for 30 minutes at 37° C. Then, cells were washed with PBS solution three times. Fluorescence changes at excitation/emission of 548/574 nm were monitored with Cytation 5 plate reader (BioTek). Mitochondrial membrane potential was also measured using JC-10 (AdipoGen, cat. 50-114-6552). Cells were cultured in 96-well black plate at a density of 5×10⁴ cells per well overnight in incubator with 5% CO₂ at 37° C. Cells were incubated with JC-10 (10 μg/ml) for 45 minutes at 37° C. Then, cells were washed with PBS solution twice. Fluorescence changes at excitation/emission of 535/595 nm for JC-10 aggregates and at 485/535 nm for JC-10 monomers were monitored with a Synergy 2 Reader (BioTek). Mitochondrial membrane potential was quantified as the fluorescence of JC-10 aggregates/monomers (595/535 nm).

Oxygen Consumption Rate assay with a Seahorse analyzer. The oxygen consumption rate (OCR) of cells was measured using the Seahorse Cell Mito Stress Test kit following the user guide (Agilent, cat. 103010-100). Briefly, cells were cultured in testing chambers at a density of 8000 cells per well overnight in incubator with 5% CO₂ at 37° C. Cells were then washed with assay medium containing Seahorse XF DMEM medium (Agilent, cat. 103575-100) with 1 mM pyruvate, 2 mM glutamine and 10 mM glucose twice, and incubated in assay medium for 1 hour in incubator without CO₂ at 37° C. Cells were treated with compounds in the order of oligomycin (1.5 μM), carbonyl cyanide-4 (trifluoromethoxy), phenylhydrazone (FCCP, 1.0 μM), and Rotenone/Antimycin (0.5 μM). The OCR of cells was monitored by using Seahorse XF HS Mini (Agilent).

Mitochondrial MPTP assay. The opening of mitochondrial permeability transition pore was measured using Invitrogen™ Image-IT™ LIVE Mitochondrial Transition Pore Assay Kit (Invitrogen, cat. I35103). Cells were cultured in 35 mm glass-bottom dishes overnight in an incubator with 5% CO₂ at 37° C. Cells were washed twice with the modified Hank's Balanced Salt Solution (HBSS, Thermo Fisher, cat. 14025092) containing 10 mM HEPES, 2 mM L-glutamine and 0.1 mM succinate (Thermo Fisher, cat. 041983.A7) and incubated with the labeling solution (1 μM Calcein, 0.2 μM MitoTracker Red, 1 mM Cobalt Chloride) for 15 minutes at 37° C. Cells were then washed with HBSS twice and imaged at excitation/emission of 494/517 nm for Calcein and at 579/599 nm for MitoTracker Red by using the Zeiss confocal microscope.

Mitochondrial Ca²⁺ retention capacity assay. The mitochondrial calcium retention capacity (CRC) was measured on a Cytation 5 reader at excitation/emission of 506/592 nm using the membrane-impermeable fluorescent probe Calcium green-5N (Invitrogen, cat. C3737). Isolated mitochondria samples (0.75 mg protein/mL) were incubated in 1 mL swelling medium supplemented with 10 mM succinate, 1 μM Calcium green-5N, and inorganic phosphate and cyclosporine A (Thermo Fisher, cat. AC457970010). One Ca²⁺ addition was 1.25 nmol (1 mL volume). Only the MPTP opening in the presence of cyclosporine A was induced by high amounts of added calcium (30 nmol Ca²⁺ in the last two additions). The CRC value was calculated as total Ca²⁺ accumulated in the mitochondria per unit (1 mg protein).

MTT assay. Cell proliferation was measured by using the MTT assay kit (Roche, cat. 11465007001). Cells were cultured in 96-well plates at a density of 2000 cells per well overnight in an incubator with 5% CO₂ at 37° C. Cells were treated with MTT labeling reagent for 4 hours at 37° C. The solubilization buffer was added to cells and then cells were incubated overnight at 37° C. Absorbance changes of the samples at 550 nm were monitored by using a Synergy 2 Reader (BioTek).

Wound healing assay. Cells were seeded into 6-well plates and cultured for 24-48 hours to reach a confluent cell monolayer. Cells were treated with serum-free medium overnight before mechanical scratching (53). Images of the wounds were taken at 0, 24 and 48 hours. Wounds areas were measured by using the wound healing plugin of ImageJ. Wound coverage %=100%×[A_{t=0 h}-A_{t=Δh}]/A_{t=0 h} (A_{t=0 h} is the area of the wound measured immediately after scratching t=0 h, A_{t=Δh} is the area of the wound measured h hours after scratch is performed).

Cell migration assay. Cell migration was measured by using Transwell assays (Corning, cat. CLS3422). Cells were cultured in Transwell inserts at a density of 1×10⁵ cells per well for 3 hours in an incubator at 37° C. with 5% CO₂. The top inserts were supplemented with DMEM medium only, and the bottom wells were supplemented with DMEM medium with 20% Fetal Bovine Serum. After incubation, the cells on the apical side of the Transwell insert membrane were removed using a cotton applicator. The cells on the bottom side of the insert were rinsed with PBS twice and fixed in 70% ethanol (Thermo Fisher, cat. R40135) for 15 minutes at room temperature. After fixation, inserts were placed into an empty well to allow the membrane to dry. Then, the insert was incubated with 0.2% crystal violet (Sigma, cat. V5265) for 5 minutes at room temperature. The insert was rinsed with water twice and images were captured by using a microscope with a 20× objective. Cell numbers were quantified using ImageJ.

TUNEL staining. The apoptosis was measured by a TUNEL assay kit (ApexBio, cat. K1134). Cells were cultured on sterile cover slips until 80% confluency and washed with PBS thrice. Then, 4% formaldehyde was applied to cells and fixed for at 4° C. 25 minutes. After fixation, cells were washed with PBS twice and incubated with 20 μM proteinase K (Invitrogen, cat. 25530049) for 5 minutes at room temperature. Then, cells were rinsed with PBS thrice and incubated in 1× equilibration buffer for 10 minutes at room temperature. Cells were stained with FITC or Cy3 labeling mix for 1 hour at 37° C. in a humidified chamber Cells were washed by PBS thrice and stained with DAPI for 5 minutes at room temperature. Cells were mounted in Fluoromount-G Anti-Fade solution and imaged at 520 nm for FITC or at 570 nm for Cy3 by using the Zeiss confocal microscope.

Caspase-3/7 activity assay. Caspase-3/7 activity was measured by using CellEvent™ Caspase-3/7 Detection Reagents (Invitrogen, cat. C10432) following the manufacturer's protocol. Specifically, cells were seeded in a 96-well black plate with a clear bottom at a density of 5×10⁴ cells per well and incubated overnight in the incubator with 5% CO₂ at 37° C. Cells were then incubated with 1× staining solution for 30 min at 37° C. Fluorescence changes at excitation/emission of 485/525 nm were monitored with a Synergy 2 Reader (BioTek).

Annexin V-FITC/Propidium Iodide (PI) apoptosis detection. Annexin V-FITC/PI apoptosis assay was performed by using the FITC Annexin V Apoptosis Detection Kit with PI (BioLegend, cat. 640914). Briefly, 1×10⁵ cells were collected in 100 μL of staining buffer. Then, cells were incubated with 5 μL of Annexin V-FITC and 2.5 μL of PI for 15 min at room temperature in the dark. Following incubation, 400 μL of binding buffer was added to the stained cells. Flow cytometry analysis of the fluorescence was performed using a Soni SH800 Cell Sorter.

Mitochondria ATP measurement via fluorescence imaging of ATP-red. BioTracker™ ATP-red dye (Millipore, cat. SCT045) is a fluorogenic indicator for ATP in mitochondria (71). Cells cultured in monolayer condition were incubated in medium with 5 μM ATP-red for 15 minutes in an incubator at 37° C. with 5% CO₂. Mitochondria were also labeled with incubating cells with 100 nM MitoTracker-Green (Invitrogen, cat. M7514) for 15 minutes to normalize their mass. Before measurement, cells were washed twice with culture medium and then fresh medium was added. Cells were imaged in a 37° C. chamber with 5% CO₂ at excitation/emission of 510/570 nm for ATP-red and at excitation/emission of 490/516 nm for MitoTracker-Green by using the Zeiss confocal microscope. The ATP-red signals could be also measured by a Synergy 2 Reader (BioTek).

Co-immunoprecipitation. Cells were lysed in the buffer containing 50 mM Tris-HCl pH 7.4, 150 mM NaCl, 10% glycerol, 1% TritonX-100, 5 mM EDTA and 1× protease inhibitor. Soluble samples were incubated with 1.5 μL ATP5α antibody at 4° C. with mixing overnight. 25 μL of protein A/G magnetic beads (Pierce, cat. 88802) were added to the co-IP samples and incubated at 4° C. with mixing overnight. Samples were washed with washing buffer thrice and then applied to SDS-PAGE analysis.

Mice and immunostaining. Animal studies were approved by the University of California, San Francisco Institutional Animal Care and Use Committee (IACUC, AN195636-01) and were performed following the guidelines of the National Institutes of Health (NIH).

For orthotopic brain tumor models, 8-to-10-week-old C57BL/6 mice (male and female in equal numbers) were used for i.e. studies. Cell lines (GL261, SB28) were suspended in DMEM for inoculation. Mice were anesthetized with isoflurane, and 30,000 tumor cells were injected orthotopically in 3 μL. Using a stereotactic frame, a burr hole was formed on the skull via a 0.7 mm drill bit 1.5 mm laterally to the right and 1.5 mm rostrally from the bregma, and a noncoring needle (26 s gauge; Hamilton) was used to inject the cells at a depth of 3 mm into the brain from the burr hole. The skin incision was sutured. Mice were then monitored daily. Mouse SB28 tumor tissue and wild-type mouse brain tissue were collected at the survival endpoint.

Frozen tissue sections were thawed at room temperature for 20 min and rinsed with PBS three times. Tissues were then fixed in 4% formaldehyde for 15 min at room temperature. After washing in PBS, tissues were permeabilized with 0.01% Triton X-100+0.1% Tween-20 for 15 min and then blocked by using 5% normal goat serum and M.O.M. blocking reagent (Vector Laboratories, cat. BMK-2202) for 1 hour at room temperature. Tissues were then incubated with primary antibodies overnight in a humidified chamber at 4° C. After washing in PBST, tissues were incubated with secondary antibodies for 1 hour. After washing again in PBST, tissues were stained with 300 nM DAPI for 5 min and mounted in Fluoromount-G Anti-Fade solution. Images were taken using a Zeiss LSM 800 confocal microscope. The primary antibodies used in the study were mouse anti-ATP5α (1:500, Abcam, cat. Ab14748), rat anti-TOMM20 (1:500, Abcam, cat. Ab289670), rabbit anti-NEMF (1:500, Proteintech, cat. 11840-1-AP), mouse anti-ANKZF1 (1:500, Santa Cruz, cat. sc-398713), chicken anti-GFP (1:500, Abeam, cat. Ab13970). The secondary antibodies were Alexa Fluor 633-, 594-, 488-conjugated secondary antibodies (1: 300, Invitrogen, cat. A21071, A11036, A32732).

Statistics. Statistical analyses were performed by using Graphpad 7.0 software. Chi-squared test and unpaired student's t-test were used for comparison. P<0.05 was considered significant, expect in gene expression analysis (FIG. 1A). *, P<0.05; **, P<0.01; ***, P<0.001; ****, P<0.0001; ns, not significant. All data were expressed as means±s.e.m.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Robichaud N, et al. Translational Control in Cancer. Cold Spring Harbor perspectives in biology. 2019; 11(7).
2. Dever T E, and Green R. The elongation, termination, and recycling phases of translation in eukaryotes. Cold Spring Harbor perspectives in biology. 2012; 4(7).
3. Kim K Q, and Zaher H S. Canary in a coal mine: collided ribosomes as sensors of cellular conditions. Trends in biochemical sciences. 2022; 47(1).
4. Brandman O, et al. A ribosome-bound quality control complex triggers degradation of nascent peptides and signals translation stress. Cell. 2012; 151(5).
5. Juszkiewicz S, and Hegde R S. Initiation of Quality Control during Poly(A) Translation Requires Site-Specific Ribosome Ubiquitination. Molecular cell. 2017; 65(4).
6. Sundaramoorthy E, et al. ZNF598 and RACK1 Regulate Mammalian Ribosome-Associated Quality Control Function by Mediating Regulatory 40S Ribosomal Ubiquitylation. Molecular cell. 2017; 65(4).
7. Hashimoto S, et al. Identification of a novel trigger complex that facilitates ribosome-associated quality control in mammalian cells. Scientific reports. 2020; 10(1).
8. Juszkiewicz S, et al. The ASC-1 Complex Disassembles Collided Ribosomes.

Molecular cell. 2020; 79(4).
9. Shao S, and Hegde R S. Reconstitution of a minimal ribosome-associated ubiquitination pathway with purified factors. Molecular cell. 2014; 55(6).
10. Shen P S, et al. Protein synthesis. Rqc2p and 60S ribosomal subunits mediate mRNA-independent elongation of nascent chains. Science (New York, N Y). 2015; 347(6217).
11. Verma R, et al. Vms1 and ANKZF1 peptidyl-tRNA hydrolases release nascent chains from stalled ribosomes. Nature. 2018; 557(7705).
12. Kostova K K, et al. CAT-tailing as a fail-safe mechanism for efficient degradation of stalled nascent polypeptides. Science (New York, N Y). 2017; 357(6349).
13. Lytvynenko I, et al. Alanine Tails Signal Proteolysis in Bacterial Ribosome-Associated Quality Control. Cell. 2019; 178(1).
14. Sitron C S, et al. Aggregation of CAT tails blocks their degradation and causes proteotoxicity in S. cerevisiae. PloS one. 2020; 15(1).
15. Choe Y J, et al. Failure of RQC machinery causes protein aggregation and proteotoxic stress. Nature. 2016; 531(7593).
16. Yonashiro R, et al. The Rqc2/Tae2 subunit of the ribosome-associated quality control (RQC) complex marks ribosome-stalled nascent polypeptide chains for aggregation. eLife. 2016; 5.
17. Wu Z, et al. MISTERMINATE Mechanistically Links Mitochondrial Dysfunction With Proteostasis Failure. Molecular cell. 2019; 75(4).
18. Li S, et al. Quality-control mechanisms targeting translationally stalled and C-terminally extended poly(GR) associated with ALS/FTD. Proc Natl Acad Sci USA. 2020; 117(40):25104-15.
19. Rimal S, et al. Inefficient quality control of ribosome stalling during APP synthesis generates CAT-tailed species that precipitate hallmarks of Alzheimer's disease. Acta neuropathologica communications. 2021; 9(1).
20. Wang N, and Wang D. Genome-wide transcriptome and translatome analyses reveal the role of protein extension and domestication in liver cancer oncogenesis. Molecular genetics and genomics: MGG. 2021; 296(3).
21. Champagne J, et al. Oncogene-dependent sloppiness in mRNA translation. Molecular cell. 2021; 81(22).
22. Rubio A, et al. Ribosome profiling reveals ribosome stalling on tryptophan codons and ribosome queuing upon oxidative stress in fission yeast. Nucleic acids research. 2021; 49(1).
23. Dango S, et al. DNA unwinding by ASCC3 helicase is coupled to ALKBH3-dependent DNA alkylation repair and cancer cell proliferation. Molecular cell. 2011; 44(3).
24. Gao J, et al. Suppression of ABCE1-Mediated mRNA Translation Limits N-MYC-Driven Cancer Progression. Cancer research. 2020; 80(17).
25. Zhou X, et al. High ANKZF1 expression is associated with poor overall survival and recurrence-free survival in colon cancer. Future oncology (London, England). 2019; 15(18).
26. Costantini S, et al. Valosin-Containing Protein (VCP)/p97: A Prognostic Biomarker and Therapeutic Target in Cancer. International journal of molecular sciences. 2021; 22(18).
27. Bi X, et al. Drosophila caliban, a nuclear export mediator, can function as a tumor suppressor in human lung cancer cells. Oncogene. 2005; 24(56).
28. Yang Q, and Gupta R. Zinc finger protein 598 inhibits cell survival by promoting U V-induced apoptosis. Oncotarget. 2017; 9(5).
29. Li G, et al. ANKZF1 knockdown inhibits glioblastoma progression by promoting intramitochondrial protein aggregation through mitoRQC. Cancer letters. 2024.
30. Gehrke S, et al. PINK1 and Parkin control localized translation of respiratory chain component mRNAs on mitochondria outer membrane. Cell metabolism. 2015; 21(1).
31. Forrest M D. Why cancer cells have a more hyperpolarised mitochondrial membrane potential and emergent prospects for therapy. bioRxiv. 2015.
32. Shi Y, et al. Gboxin is an oxidative phosphorylation inhibitor that targets glioblastoma. Nature. 2019; 567(7748).
33. Guieze R, et al. Mitochondrial Reprogramming Underlies Resistance to BCL-2 Inhibition in Lymphoid Malignancies. Cancer cell. 2019; 36(4).
34. Ramamoorthy M D, et al. Reserpine Induces Apoptosis and Cell Cycle Arrest in Hormone Independent Prostate Cancer Cells through Mitochondrial Membrane Potential Failure.

Anti-cancer agents in medicinal chemistry. 2018; 18(9).
35. Heerdt B G, et al. Growth properties of colonic tumor cells are a function of the intrinsic mitochondrial membrane potential. Cancer research. 2006; 66(3).
36. Zorova L D, et al. Mitochondrial membrane potential. Analytical biochemistry. 2018; 552.
37. Maria A, et al. Biochemistry, Electron Transport Chain. StatPearls Publishing; 2020.
38. Liberti M V, and Locasale J W. The Warburg Effect: How Does it Benefit Cancer Cells? Trends in biochemical sciences. 2016; 41(3).
39. Varn F S, et al. Glioma progression is shaped by genetic evolution and microenvironment interactions. Cell. 2022; 185(12).
40. Rutka J T, et al. Establishment and characterization of five cell lines derived from human malignant gliomas. Acta neuropathologica. 1987; 75(1).
41. Kim E, et al. Phosphorylation of EZH2 activates STAT3 signaling via STAT3 methylation and promotes tumorigenicity of glioblastoma stem-like cells. Cancer cell. 2013; 23(6).
42. Sonoda Y, et al. Formation of intracranial tumors by genetically modified human astrocytes defines four pathways critical in the development of human anaplastic astrocytoma. Cancer research. 2001; 61(13).
43. Saito S, et al. The Hbs1-Dom34 protein complex functions in non-stop mRNA decay in mammalian cells. The Journal of biological chemistry. 2013; 288(24).
44. Osuna B A, et al. In vitro analysis of RQC activities provides insights into the mechanism and function of CAT tailing. eLife. 2017; 6.
45. Morgenstern M, et al. Definition of a High-Confidence Mitochondrial Proteome at Quantitative Scale. Cell reports. 2017; 19(13).
46. Chang Y W, et al. Spatial and temporal dynamics of ATP synthase from mitochondria toward the cell surface. Communications biology. 2023; 6(1).
47. Alavian K N, et al. An uncoupling channel within the c-subunit ring of the F1FO ATP synthase is the mitochondrial permeability transition pore. Proceedings of the National Academy of Sciences of the United States of America. 2014; 111(29).
48. Giorgio V, et al. Dimers of mitochondrial ATP synthase form the permeability transition pore. Proceedings of the National Academy of Sciences of the United States of America. 2013; 110(15).
49. Bonora M, et al. Role of the c subunit of the FO ATP synthase in mitochondrial permeability transition. Cell cycle (Georgetown, Tex). 2013; 12(4).
50. Ichas F, et al. Mitochondria are excitable organelles capable of generating and conveying electrical and calcium signals. Cell. 1997; 89(7).
51. Stockert J C, et al. Tetrazolium salts and formazan products in Cell Biology: Viability assessment, fluorescence imaging, and labeling perspectives. Acta histochemica. 2018; 120(3).
52. Justus C R, et al. In vitro cell migration and invasion assays. Journal of visualized experiments: JoVE. 2014(88).
53. Grada A, et al. Research Techniques Made Simple: Analysis of Collective Cell Migration Using the Wound Healing Assay. The Journal of investigative dermatology. 2017; 137(2).
54. Mashimo M, et al. The 89-kDa PARP1 cleavage fragment serves as a cytoplasmic PAR carrier to induce AIF-mediated apoptosis. The Journal of biological chemistry. 2021; 296.
55. Comelli M, et al. FIFO ATP Synthase Is Expressed at the Surface of Embryonic Rat Heart-Derived H9c2 Cells and Is Affected by Cardiac-Like Differentiation. Journal of cellular biochemistry. 2016; 117(2).
56. Lv L, et al. NEMF-mediated Listerin-independent mitochondrial translational surveillance by E3 ligase Pirh2 and mitochondrial protease ClpXP. Cell reports. 2024; 43(3).
57. Cormio A, et al. Mitochondrial Caseinolytic Protease P: A Possible Novel Prognostic Marker and Therapeutic Target in Cancer. International journal of molecular sciences. 2021; 22(12).
58. Ventura-Clapier R, et al. Transcriptional control of mitochondrial biogenesis: the central role of PGC-1alpha. Cardiovascular research. 2008; 79(2).
59. Wu Z, et al. Mechanisms controlling mitochondrial biogenesis and respiration through the thermogenic coactivator PGC-1. Cell. 1999; 98(1).
60. Endlicher R, et al. The Mitochondrial Permeability Transition Pore-Current Knowledge of Its Structure, Function, and Regulation, and Optimized Methods for Evaluating Its Functional State. Cells. 2023; 12(9).
61. Beutner G, et al. Complexes between porin, hexokinase, mitochondrial creatine kinase and adenylate translocator display properties of the permeability transition pore. Implication for regulation of permeability transition by the kinases. Biochimica et biophysica acta. 1998; 1368(1).
62. Baines C P, et al. Voltage-dependent anion channels are dispensable for mitochondrial-dependent cell death. Nature cell biology. 2007; 9(5).
63. Gutiérrez-Aguilar M, et al. Genetic manipulation of the cardiac mitochondrial phosphate carrier does not affect permeability transition. Journal of molecular and cellular cardiology. 2014; 72.
64. Kokoszka J E, et al. The ADP/ATP translocator is not essential for the mitochondrial permeability transition pore. Nature. 2004; 427(6973).
65. Karch J, et al. Inhibition of mitochondrial permeability transition by deletion of the ANT family and CypD. Science advances. 2019; 5(8).
66. Beutner G, et al. The Mitochondrial Permeability Transition Pore and ATP Synthase. Handbook of experimental pharmacology. 2017; 240.
67. Petronilli V, et al. Regulation of the permeability transition pore, a voltage-dependent mitochondrial channel inhibited by cyclosporin A. Biochimica et biophysica acta. 1994; 1187(2).
68. Boyman L, et al. Dynamics of the mitochondrial permeability transition pore: Transient and permanent opening events. Archives of biochemistry and biophysics. 2019; 666.
69. Antoniel M, et al. The unique histidine in OSCP subunit of F-ATP synthase mediates inhibition of the permeability transition pore by acidic pH. EMBO reports. 2018; 19(2).
70. Carraro M, et al. The Unique Cysteine of F-ATP Synthase OSCP Subunit Participates in Modulation of the Permeability Transition Pore. Cell reports. 2020; 32(9).
71. Wang L, et al. A Multisite-Binding Switchable Fluorescent Probe for Monitoring Mitochondrial ATP Level Fluctuation in Live Cells. Angewandte Chemie (International ed in English). 2016; 55(5).

What is claimed is:

1. A method for detecting presence of cancer cells in a human tissue sample, the method comprising:
obtaining, or having obtained, a human tissue sample with cancer cells; and
detecting in the human tissue sample for abnormalities in ribosome-associated quality control by detecting the presence of mitochondrial stress-induced protein carboxyl-terminal terminal alanine and threonine tailing (msiCAT-tailing) proteins, wherein the presence of msiCAT-tailing is indicative of the presence of cancer stem cells.

2. The method of claim 1, wherein the cancer stem cells are glioma, glioblastoma, medulloblastoma, oligodendroglioma, ependymoma stem cells, or combinations thereof.

3. The method of claim 1, further comprising detecting an increase in expression of at least one of: ABCE1, ASCC1, ASCC2, ASCC3, RACK1, or VCP.

4. The method of claim 1, further comprising detecting a decrease in expression of ANKZF1.

5. The method of claim 1, wherein the msiCAT-tailing proteins are msiCAT-tailed mitochondrial proteins are selected from C-I30, COX4 (cytochrome c oxidase subunit 4), and ATP5α (ATP synthase F1 subunit alpha).

6. The method of claim 5, further comprising detecting an increase in expression or post-translational modification of nuclear export mediator factor (NEMF), or both.

7. The method of claim 1, wherein the msiCAT-tailing proteins are msiCAT-tailed mitochondrial proteins detected by at least one of:
measuring a presence of detergent insoluble aggregates;
measuring a presence of oligomers;
measuring msiCAT-tailing modification of mitochondrial ATP synthase F1 subunit alpha (ATP5α);
measuring an increase in cytoplasmic ATP5α-AT20 near mitochondria and forming protein aggregates;
measuring a higher mitochondrial membrane potential;
measuring a reduced ATP production;
measuring a reduction in open mitochondrial permeability transition pores (MPTP);
measuring overexpression of short AT repeat tail (AT3);
measuring overexpression of long AT repeat tail (AT20) fused with ATP5α; or
determining an increase in a ratio of cells that have aggregates versus cells that do not have aggregates.

8. The method of claim 1, further comprising measuring a rate of protein translation in the cancer cells and detecting a decrease in the rate of protein translation, wherein a decrease in the rate of protein translation is indicative of the presence of glioma, glioblastoma, medulloblastoma, oligodendroglioma, or ependymoma stem cells, or combinations thereof, in the human tissue sample.

9. The method of claim 1, further comprising measuring ATP5α-AT20 proteins, wherein an increase in expression of ATP5α-AT20 proteins is indicative of the presence of glioblastoma stem cells in the human tissue sample.

10. The method of claim 1, further comprising determining if glioblastoma cancer cells are resistant to staurosporine (STS), temozolomide (TMZ), or both.

11. A method for treating a mammal having a cancer with abnormalities in ribosome-associated quality control, wherein the method comprises:
identifying in the cancer one or more abnormalities in ribosome-associated quality control by detecting a presence of mitochondrial stress-induced protein carboxyl-terminal terminal alanine and threonine tailing (msiCAT-tailing); and
administering a cancer treatment to the mammal having the cancer, wherein the cancer treatment is selected from the group consisting of a nucleic acid msiCAT-tailing inhibitor or an msiCAT-tailing antagonist, in an amount effective to reduce the presence of msiCAT-tailing.

12. The method of claim 11, wherein the cancer is glioblastoma.

13. The method of claim 11, wherein the msiCAT-tailing antagonist is selected from at least one of: anisomycin, puromycin, borrelidin, isomers and derivatives thereof, and combinations of the same.

14. The method of claim 11, wherein the msiCAT-tailing antagonist is selected from anisomycin, isomers and derivatives thereof.

15. The method of claim 11, wherein the nucleic acid msiCAT-tailing inhibitor at least one of: knocks down NEMF (sgNEMF) expression, or increase the expression of ANKZF1 (oeANZKF1).

16. The method of claim 11, wherein the cancer is resistant to staurosporine (STS), temozolomide (TMZ), or both.

17. The method of claim 11, wherein the cancer treatment reduces migration of glioma, glioblastoma, medulloblastoma, oligodendroglioma, or ependymoma stem cells, or combinations thereof.

18. A method for treating a cancer that has become resistant to staurosporine (STS), temozolomide (TMZ), or both, wherein the method comprises:
administering a mitochondrial stress-induced protein carboxyl-terminal terminal alanine and threonine tailing (msiCAT-tailing) antagonist in an amount sufficient to render the cancer susceptible to the staurosporine (STS), temozolomide (TMZ), or both.

19. The method of claim 18, wherein the cancer is glioblastoma.

20. The method of claim 18, wherein the msiCAT-tailing antagonist is selected from at least one of: anisomycin, puromycin, borrelidin, isomers and derivatives thereof, and combinations of the same.

21. The method of claim 18, wherein the msiCAT-tailing antagonist is selected from anisomycin, isomers and derivatives thereof.

22. The method of claim 18, wherein the msiCAT-tailing antagonist is a nucleic acid msiCAT-tailing inhibitor that at least one of: knocks down NEMF (sgNEMF) expression, or increase the expression of ANKZF1 (oeANZKF1).

23. A method for treating a cancer with abnormalities in ribosome-associated quality control, wherein the method comprises administering a cancer treatment to a mammal having cancer and identified as having cancer cells are resistant to staurosporine (STS), temozolomide (TMZ), or both, comprising:
obtaining, or having obtained, a human tissue sample with cancer cells having the abnormalities in ribosome-associated quality control;
detecting in the human tissue sample for abnormalities in ribosome-associated quality control by detecting a presence of mitochondrial stress-induced protein carboxyl-terminal terminal alanine and threonine tailing (msiCAT-tailing) proteins, wherein the presence of msiCAT-tailing is indicative of the presence of glioblastoma stem cells; and
administering a cancer treatment to the mammal having cancer, wherein the cancer treatment is selected from the group consisting of a nucleic acid msiCAT-tailing inhibitor or an msiCAT-tailing antagonist, in an amount effective to reduce the presence of msiCAT-tailing.

24. The method of claim 23, wherein the cancer is glioma, glioblastoma, medulloblastoma, oligodendroglioma, ependymoma, or combinations thereof.

25. The method of claim 23, wherein the msiCAT-tailing antagonist is selected from at least one of: anisomycin, puromycin, borrelidin, isomers and derivatives thereof, and combinations of the same.

26. The method of claim 23, wherein the msiCAT-tailing antagonist is selected from anisomycin, isomers and derivatives thereof.

27. The method of claim 23, wherein the nucleic acid msiCAT-tailing inhibitor at least one of: knocks down NEMF (sgNEMF) expression, or increase the expression of ANKZF1 (oeANZKF1).

28. The method of claim 23, wherein the cancer cells are resistant to staurosporine (STS), temozolomide (TMZ), or both, and the msiCAT-tailing inhibitor increases susceptibility of the cancer cells to STS or MTZ treatment.

29. The method of claim 23, wherein the treatment reduces migration of glioma, glioblastoma, medulloblastoma, oligodendroglioma, or ependymoma stem cells, or combinations thereof.
